(19) United States
(12) Patent Application Publication
(10) Pub. No.: US 2025/0022127 A1
(43) Pub. Date: Jan. 16, 2025

(54) MEDICAL SUPPORT DEVICE, ENDOSCOPE APPARATUS, MEDICAL SUPPORT METHOD, AND PROGRAM

(71) Applicant: FUJIFILM Corporation, Tokyo (JP)

(72) Inventor: Masaaki OOSAKE, Kanagawa (JP)

(73) Assignee: FUJIFILM Corporation, Tokyo (JP)

(21) Appl. No.: 18/674,933

(22) Filed: May 27, 2024

(30) Foreign Application Priority Data
Jul. 12, 2023 (JP) 2023-114767

(51) Int. Cl. G06T 7/00; G06T 7/11; G06V 10/44; G06V 10/74; G16H 30/20; G16H 30/40

(52) U.S. Cl. CPC G06T 7/0012; G06T 7/11; G06V 10/44; G06V 10/761; G16H 30/20; G16H 30/40; G06T 2207/10068; G06T 2207/30096; G06V 2201/07

(57) ABSTRACT

A medical support device acquires a first medical image and a second medical image while an endoscope is inserted into a luminal organ. A processor outputs the first medical image and/or the second medical image in a case where a similarity between a first partial region and a second partial region exceeds a threshold value. The output first medical image is an image represented in an aspect in which the first partial region and a first other region are discriminable from each other. The output second medical image is an image represented in an aspect in which the second partial region and a second other region are discriminable from each other.

### CROSS-REFERENCE TO RELATED APPLICATIONS

This application claims priority under 35 USC 119 from Japanese Patent Application No. 2023-114767 filed on Jul. 12, 2023, the disclosure of which is incorporated by reference herein.

### BACKGROUND OF THE INVENTION

### 1. Technical Field

The present disclosure relates to a medical support device, an endoscope apparatus, a medical support method, and a program.

### 2. Related Art

WO2019/167623A discloses an image processing apparatus including an image input unit, a parameter calculation unit, and a display controller. In the image processing apparatus described in WO2019/167623A, the image input unit inputs a first image and a second image captured at different points in time. In addition, the image input unit inputs the first image captured by first observation light and the second image captured by second observation light different from the first observation light. The parameter calculation unit calculates a parameter for performing registration between the first image and the second image. The image generation unit applies the parameter to the first image to generate a registration first image. The display controller sequentially displays the input first image and the generated registration first image on a display device.

JP2017-097836A discloses an image processing apparatus comprising an image acquisition unit, a recognition unit, and a matching unit. In the image processing apparatus disclosed in JP2017-097836A, the image acquisition unit acquires a first image and a second image each including a specific site in a subject, which are captured at different timings from each other in time series. The recognition unit recognizes the specific site in each image acquired by the image acquisition unit, and recognizes a plurality of first specific site candidates in the first image and a plurality of second specific site candidates in the second image. The matching unit performs matching between each of the plurality of first specific site candidates and each of the plurality of second specific site candidates based on a difference between a feature amount of each of the plurality of first specific site candidates and a feature amount of each of the plurality of second specific site candidates to specify a correspondence relationship representing the same specific part in the first image and the second image.

JP2016-209336A discloses a magnetic resonance imaging apparatus comprising a magnetic field generation unit, a detection unit, and a processing unit. In the magnetic resonance imaging device disclosed in JP2016-209336A, the magnetic field generation unit generates a uniform static magnetic field and a gradient magnetic field superimposed on the static magnetic field in a space accommodating a subject. The detection unit detects a nuclear magnetic resonance signal generated from the subject by irradiating the subject with a high-frequency magnetic field. The processing unit images the detected nuclear magnetic resonance signal. In addition, the processing unit acquires past examination information from a past image obtained at a different date and time or obtained from a different apparatus, determines current examination information by using the acquired examination information, and displays a current image acquired based on the determined examination information and the past image in a comparable state.

### SUMMARY

One embodiment according to the present disclosure provides a medical support device, an endoscope apparatus, a medical support method, and a program that can contribute to allowing a user or the like to thoroughly observe again, via an endoscope, a region observed in a luminal organ via the endoscope by the user or the like while the endoscope is inserted into the luminal organ.

A first aspect according to the present disclosure is a medical support device comprising a processor, in which the processor is configured to: acquire a first medical image obtained by imaging an inside of a luminal organ with an endoscope and a second medical image obtained temporally later than the first medical image by imaging the inside of the luminal organ with the endoscope while the endoscope is inserted into the luminal organ; and execute a first output process of outputting the first medical image and/or a second output process of outputting the second medical image in a case where a similarity between a first partial region that is a part of the first medical image and a second partial region that is a part of the second medical image exceeds a threshold value, the first medical image output by executing the first output process is an image represented in an aspect in which the first partial region and a first other region that is another region in the first medical image are discriminable from each other, and the second medical image output by executing the second output process is an image represented in an aspect in which the second partial region and a second other region that is another region in the second medical image are discriminable from each other.

A second aspect according to the present disclosure is the medical support device according to the first aspect, in which the first medical image output by executing the first output process is an image in which the first partial region is emphasized more than the first other region, and the second medical image output by executing the second output process is an image in which the second partial region is emphasized more than the second other region.

A third aspect according to the present disclosure is the medical support device according to the first or second aspect, in which the first medical image is an image obtained in an insertion step in which the endoscope is inserted into the luminal organ, and the second medical image is an image obtained in a removal step in which the endoscope is removed from the luminal organ.

A fourth aspect according to the present disclosure is the medical support device according to any one of the first to third aspects, in which the first partial region is at least one of a plurality of first divided regions obtained by dividing the first medical image according to a first rule, and the second partial region is at least one of a plurality of second divided regions obtained by dividing the second medical image according to the first rule.

A fifth aspect according to the present disclosure is the medical support device according to the fourth aspect, in which the processor is further configured to: derive a map showing a distribution of the similarities between the plurality of first divided regions and the plurality of second divided regions based on the first medical image and the second medical image; and extract, from the plurality of first divided regions and the plurality of second divided regions, the first divided region and the second divided region in which the similarity exceeds the threshold value according to the derived map, as the first partial region and the second partial region in which the similarity exceeds the threshold value.

A sixth aspect according to the present disclosure is the medical support device according to the fifth aspect, in which the processor derives the map by using AI.

A seventh aspect according to the present disclosure is the medical support device according to the fifth or sixth aspect, in which the map is derived according to a first instruction given from an outside.

An eighth aspect according to the present disclosure is the medical support device according to any one of the first to third aspects, in which the first partial region is at least one of a plurality of third divided regions obtained by dividing a first region that is a part of the first medical image according to a second rule, and the second partial region is at least one of a plurality of fourth divided regions obtained by dividing a second region that is a part of the second medical image according to the second rule.

A ninth aspect according to the present disclosure is the medical support device according to the eighth aspect, in which the processor is further configured to: derive the similarity between each of the plurality of third divided regions and each of the plurality of fourth divided regions based on each of the plurality of third divided regions and each of the plurality of fourth divided regions; and extract, from the plurality of third divided regions and the plurality of fourth divided regions, the third divided region and the fourth divided region in which the similarity exceeds the threshold value as the first partial region and the second partial region in which the similarity exceeds the threshold value.

A tenth aspect according to the present disclosure is the medical support device according to the ninth aspect, in which the processor derives the similarity by using AI.

An eleventh aspect according to the present disclosure is the medical support device according to the tenth aspect, in which the similarity is derived according to a second instruction given from an outside.

A twelfth aspect according to the present disclosure is the medical support device according to any one of the eighth to eleventh aspects, in which each of the first region and the second region is a feature region recognized by performing an object recognition process as a region having features determined in advance.

A thirteenth aspect according to the present disclosure is the medical support device according to the twelfth aspect, in which the feature region is a region in which a lesion is shown, a marked region, a region in which an organ is shown, and/or a region in which a treatment tool is shown.

A fourteenth aspect according to the present disclosure is the medical support device according to the twelfth or thirteenth aspect, in which object recognition AI is used in the object recognition process.

A fifteenth aspect according to the present disclosure is the medical support device according to any one of the twelfth to fourteenth aspects, in which the first medical image is stored in a first storage region on a condition that the first region is recognized as the feature region by performing the object recognition process.

A sixteenth aspect according to the present disclosure is the medical support device according to any one of the first to fifteenth aspects, in which the first medical image is stored in a second storage region in response to a third instruction given from an outside.

A seventeenth aspect according to the present disclosure is the medical support device according to any one of the first to sixteenth aspects, in which the first medical image and the second medical image are images obtained by imaging a region irradiated with the same type of light in the luminal organ, by the endoscope.

An eighteenth aspect according to the present disclosure is the medical support device according to any one of the first to seventeenth aspects, in which the first medical image output by executing the first output process is displayed on a first screen, and the second medical image output by executing the second output process is displayed on a second screen.

A nineteenth aspect according to the present disclosure is a medical support device comprising a processor, in which the processor is configured to: acquire a first medical image obtained by imaging an inside of a luminal organ with an endoscope and a second medical image obtained temporally later than the first medical image by imaging the inside of the luminal organ with the endoscope while the endoscope is inserted into the luminal organ; and execute a first output process of outputting the first medical image and/or a second output process of outputting the second medical image in a case where a difference between a first partial region that is a part of the first medical image and a second partial region that is a part of the second medical image is less than a threshold value, the first medical image output by executing the first output process is an image represented in an aspect in which the first partial region and a first other region that is another region in the first medical image are discriminable from each other, and the second medical image output by executing the second output process is an image represented in an aspect in which the second partial region and a second other region that is another region in the second medical image are discriminable from each other.

A twentieth aspect according to the present disclosure is an endoscope apparatus comprising: the medical support device according to any one of the first to nineteenth aspects; and the endoscope.

A twenty-first aspect according to the present disclosure is a medical support method comprising: acquiring a first medical image obtained by imaging an inside of a luminal organ with an endoscope and a second medical image obtained temporally later than the first medical image by imaging the inside of the luminal organ with the endoscope while the endoscope is inserted into the luminal organ; and executing a first output process of outputting the first medical image and/or a second output process of outputting the second medical image in a case where a similarity between a first partial region that is a part of the first medical image and a second partial region that is a part of the second medical image exceeds a threshold value, in which the first medical image output by executing the first output process is an image represented in an aspect in which the first partial region and a first other region that is another region in the first medical image are discriminable from each other, and the second medical image output by executing the second output process is an image represented in an aspect in which the second partial region and a second other region that is another region in the second medical image are discriminable from each other.

A twenty-second aspect according to the present disclosure is a program for causing a computer to execute a medical support process, the medical support process comprising: acquiring a first medical image obtained by imaging an inside of a luminal organ with an endoscope and a second medical image obtained temporally later than the first medical image by imaging the inside of the luminal organ with the endoscope while the endoscope is inserted into the luminal organ; and executing a first output process of outputting the first medical image and/or a second output process of outputting the second medical image in a case where a similarity between a first partial region that is a part of the first medical image and a second partial region that is a part of the second medical image exceeds a threshold value, in which the first medical image output by executing the first output process is an image represented in an aspect in which the first partial region and a first other region that is another region in the first medical image are discriminable from each other, and the second medical image output by executing the second output process is an image represented in an aspect in which the second partial region and a second other region that is another region in the second medical image are discriminable from each other.

### BRIEF DESCRIPTION OF THE DRAWINGS

Exemplary embodiments of the technology of the disclosure will be described in detail based on the following figures, wherein:

FIG. 1 is a conceptual diagram showing an example of an aspect in which an endoscope apparatus is used;

FIG. 2 is a conceptual diagram showing an example of an overall configuration of the endoscope apparatus;

FIG. 3 is a block diagram showing an example of a hardware configuration of an electric system of the endoscope apparatus;

FIG. 4 is a block diagram showing an example of functions of main units of a processor included in a medical support device and an example of information stored in a storage;

FIG. 5 is a conceptual diagram showing an example of processing contents of a recognition unit and a controller;

FIG. 6 is a conceptual diagram showing an example of processing contents of the recognition unit and the controller in an insertion step;

FIG. 7 is a conceptual diagram showing an example of processing contents of the recognition unit and the controller in a removal step;

FIG. 8 is a conceptual diagram showing an example of processing contents for deriving a similarity between a lesion shown in an insertion step frame and a lesion shown in a removal step frame;

FIG. 9 is a conceptual diagram showing an example of processing contents using the removal step frame and a map by the controller and processing contents using the insertion step frame and the map by the controller in a case where a high similarity distribution region is obtained;

FIG. 10 is a conceptual diagram showing an example of processing contents using an insertion tag-assigned frame and a removal tag-assigned frame by the controller;

FIG. 11A is a flowchart showing an example of a flow of a medical support process.

FIG. 11B is a continuation of the flowchart shown in FIG. 11A;

FIG. 12 is a conceptual diagram showing a first modification example of processing contents by the controller according to a first embodiment;

FIG. 13 is a conceptual diagram showing a second modification example of the processing contents by the controller according to the first embodiment;

FIG. 14 is a conceptual diagram showing a third modification example of the processing contents by the controller according to the first embodiment;

FIG. 15 is a conceptual diagram showing a fourth modification example of the processing contents by the controller according to the first embodiment;

FIG. 16 is a conceptual diagram showing a fifth modification example of the processing contents by the controller according to the first embodiment;

FIG. 17 is a conceptual diagram showing a sixth modification example of the processing contents by the controller according to the first embodiment;

FIG. 18 is a conceptual diagram showing an example of processing contents of the recognition unit and the controller in the insertion step in a case where an endoscope apparatus according to a second embodiment is used;

FIG. 19 is a conceptual diagram showing an example of processing contents for deriving a similarity between a lesion included in an insertion step frame and a lesion included in a removal step frame in a case where the endoscope apparatus according to the second embodiment is used;

FIG. 20 is a conceptual diagram showing an example of processing contents using a removal step lesion region and the map by the controller and processing contents using an insertion step lesion region and the map by the controller in a case where the high similarity distribution region is obtained by using the endoscope apparatus according to the second embodiment; and

FIG. 21 is a conceptual diagram showing an example of a series of pieces of processing in which a processor included in a computer gives a processing execution request to an external device via a network, the external device executes processing according to the processing execution request, and the processor included in the computer receives a processing result from the external device.

### DETAILED DESCRIPTION

Hereinafter, examples of embodiments of a medical support device, an endoscope apparatus, a medical support method, and a program according to the present disclosure will be described with reference to the accompanying drawings.

First, the wording used in the following description will be described.

CPU is an abbreviation for a “central processing unit”. GPU is an abbreviation for a “graphics processing unit”. GPGPU is an abbreviation for a “general-purpose computing on graphics processing units”. APU is an abbreviation for an “accelerated processing unit”. TPU is an abbreviation for a “tensor processing unit”. RAM is an abbreviation for a “random access memory”. NVM is an abbreviation for a “non-volatile memory”. EEPROM is an abbreviation for an “electrically erasable programmable read-only memory”. ASIC is an abbreviation for an “application specific integrated circuit”. PLD is an abbreviation for a “programmable logic device”. FPGA is an abbreviation for a “field-programmable gate array”. SoC is an abbreviation for a “system-on-a-chip”. SSD is an abbreviation for a “solid state drive”. USB is an abbreviation for a “universal serial bus”. HDD is an abbreviation for a “hard disk drive”. EL is an abbreviation for “electro-luminescence”. CMOS is an abbreviation for a “complementary metal oxide semiconductor”. CCD is an abbreviation for a “charge coupled device”. AI is an abbreviation for “artificial intelligence”. BLI is an abbreviation for “blue light imaging”. LCI is an abbreviation for “linked color imaging”. I/F is an abbreviation for an “Interface”. SSL stands for a “sessile serrated lesion”. LAN is an abbreviation for a “local area network”. WAN is an abbreviation for a “wide area network”. 5G is an abbreviation for a “5th generation mobile communication system”.

In the following description, a processor with a reference (hereinafter, simply referred to as a “processor”) may be one computing device or a combination of a plurality of computing devices. In addition, the processor may be one type of a computing device or a combination of a plurality of types of computing devices. Examples of the computing device include a CPU, a GPU, a GPGPU, an APU, or a TPU.

In the following description, a memory with a reference is a memory such as a RAM in which information is temporarily stored, and is used as a work memory by the processor.

In the following description, a storage with a reference is one or a plurality of non-volatile storage devices that store various programs, various parameters, and the like. Examples of the non-volatile storage device include a flash memory, a magnetic disk, or a magnetic tape. In addition, other examples of the storage include a cloud storage.

In the following embodiment, an external I/F with a reference transmits and receives various types of information between a plurality of devices connected to each other. Examples of the external I/F include a USB interface. A communication I/F including a communication processor, an antenna, and the like may be applied to the external I/F. The communication I/F performs communication between a plurality of computers. Examples of a communication standard applied to the communication I/F include a wireless communication standard including 5G, Wi-Fi (registered trademark), or Bluetooth (registered trademark).

In the following embodiments, “A and/or B” is synonymous with “at least one of A or B”. That is, “A and/or B” means that it may be only A, only B, or a combination of A and B. In addition, in the present specification, in a case where three or more matters are associated and represented by “and/or”, the same concept as “A and/or B” is applied.

### First Embodiment

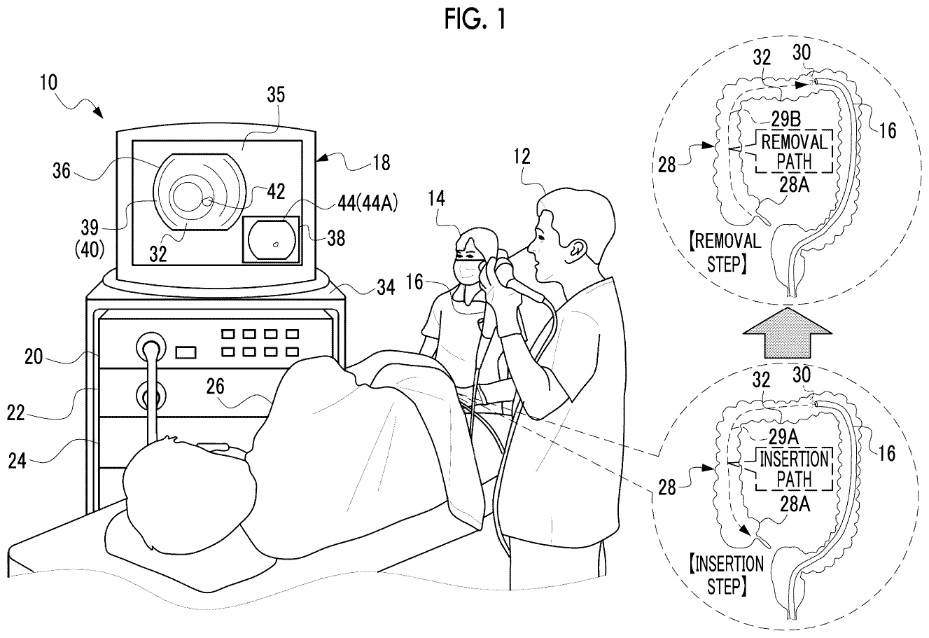

FIG. 1 is a conceptual diagram showing an example of an aspect in which an endoscope apparatus 10 is used. As shown in FIG. 1, the endoscope apparatus 10 is used by a doctor 12 in an endoscopy or the like. The endoscopy is assisted by a staff such as a nurse 14.

The endoscope apparatus 10 is connected to a communication device (not shown) in a communicable manner, and information obtained by the endoscope apparatus 10 is transmitted to the communication device. Examples of the communication device include a server, a personal computer, and/or a tablet terminal that manage various types of information such as an electronic medical record. The communication device receives the information transmitted from the endoscope apparatus 10 and executes a process using the received information (for example, a process of storing the information in an electronic medical record or the like).

The endoscope apparatus 10 comprises an endoscope 16, a display device 18, a light source device 20, a control device 22, and a medical support device 24. In the first embodiment, the endoscope apparatus 10 is an example of an “endoscope apparatus” according to the present disclosure, and the endoscope 16 is an example of an “endoscope” according to the present disclosure.

The endoscope apparatus 10 is a modality for performing medical care on a large intestine 28 included in a body of a subject 26 (for example, a patient) by using the endoscope 16. In the first embodiment, the large intestine 28 is a target to be observed by the doctor 12.

The endoscope 16 is used by the doctor 12 and is inserted into a luminal organ of the subject 26. In the first embodiment, the endoscope 16 is inserted into the large intestine 28 of the subject 26. The large intestine 28 is an example of a “luminal organ” according to the present disclosure.

The endoscope apparatus 10 causes the endoscope 16 inserted into the large intestine 28 of the subject 26 to image the inside of the large intestine 28 of the subject 26 and performs various medical treatments on the large intestine 28 as necessary.

The endoscope apparatus 10 acquires and outputs an image showing an aspect in the large intestine 28 by imaging the inside of the large intestine 28 of the subject 26. In the first embodiment, the endoscope apparatus 10 is an endoscope apparatus having an optical imaging function of capturing reflected light obtained by being reflected by an intestinal wall 32 of the large intestine 28 by irradiating the inside of the large intestine 28 with light 30.

Although the endoscopy of the large intestine 28 is illustrated here, this is merely an example, and the present disclosure is established even in a case of an endoscopy of a luminal organ such as an esophagus, a stomach, a duodenum, or a trachea.

The light source device 20, the control device 22, and the medical support device 24 are installed on a wagon 34. A plurality of tables are provided in the wagon 34 along a vertical direction, and the medical support device 24, the control device 22, and the light source device 20 are installed from a lower table to an upper table. In addition, the display device 18 is installed on the uppermost table in the wagon 34.

The control device 22 controls the entire endoscope apparatus 10. The medical support device 24 performs various types of image processing on an image obtained by imaging the intestinal wall 32 with the endoscope 16 under the control of the control device 22.

The display device 18 displays various types of information including the image. Examples of the display device 18 include a liquid crystal display or an EL display. In addition, a tablet terminal with a display may be used instead of the display device 18 or together with the display device 18.

A screen 35 is displayed on the display device 18. The screen 35 includes a plurality of display regions. The plurality of display regions are arranged side by side in the screen 35. In the example shown in FIG. 1, a first display region 36 and a second display region 38 are shown as an example of the plurality of display regions. A size of the first display region 36 is larger than a size of the second display region 38. The first display region 36 is used as a main display region, and the second display region 38 is used as a sub-display region. A size relationship between the first display region 36 and the second display region 38 is not limited to this, and need only be a size relationship that fits on the screen 35. In the first embodiment, the first display region 36 is an example of a “second screen” according to the present disclosure, and the second display region 38 is an example of a “first screen” according to the present disclosure.

An endoscopic moving image 39 is displayed in the first display region 36. The endoscopic moving image 39 is a moving image acquired by imaging the intestinal wall 32 with the endoscope 16 inside the large intestine 28 of the subject 26. In the example shown in FIG. 1, as an example of the endoscopic moving image 39, a moving image in which the intestinal wall 32 is shown is shown.

The intestinal wall 32 shown in the endoscopic moving image 39 includes a lesion 42 (for example, one lesion 42 in the example shown in FIG. 1,) as a region of interest (that is, an observation target region) that is watched by the doctor 12, and the doctor 12 can visually recognize an aspect of the intestinal wall 32 including the lesion 42 through the endoscopic moving image 39. In the first embodiment, the lesion 42 is an example of a “lesion” according to the present disclosure.

The lesion 42 has various types, and examples of the type of the lesion 42 include a neoplastic polyp and a non-neoplastic polyp. Examples of the type of the neoplastic polyp include an adenomatous polyp (for example, SSL). Examples of the type of the non-neoplastic polyp include a hamartomatous polyp, a hyperplastic polyp, and an inflammatory polyp. The types illustrated here are types assumed in advance as the types of the lesion 42 in a case where the endoscopy is performed on the large intestine 28, and the types of the lesion 42 may be different depending on the organ on which the endoscopy is performed.

In the first embodiment, for convenience of description, a form example is described in which one lesion 42 is shown in the endoscopic moving image 39, but the present disclosure is not limited to this. The present disclosure is established even in a case where a plurality of the lesions 42 are shown in the endoscopic moving image 39.

In the first embodiment, the lesion 42 is illustrated, but this is merely an example. The region of interest (that is, the observation target region) that is watched by the doctor 12 may be an organ (for example, a duodenal papilla), a mark, an artificial treatment tool (for example, an artificial clip), a treated region (for example, a region in which a trace of removal of a polyp or the like remains), or the like.

The image displayed in the first display region 36 is one frame 40 included in a moving image configured to include a plurality of frames 40 along a time series. That is, a plurality of frames 40 along the time series are displayed in the first display region 36 at a predetermined frame rate (for example, several tens of frames/second).

Examples of the moving image displayed in the first display region 36 include a moving image of a live view method. The live view method is only an example, and a moving image which is temporarily stored in a memory or the like and then is displayed, such as a moving image of a post view method, may be employed. In addition, each frame included in a recording moving image stored in a memory or the like may be reproduced and displayed on the screen 35 (for example, the first display region 36) as the endoscopic moving image 39.

In the screen 35, the second display region 38 is adjacent to the first display region 36 and is displayed in the lower right in the screen 35 in front view. A display position of the second display region 38 may be anywhere in the screen 35 of the display device 18. However, the display position is preferably displayed at a position comparable to the endoscopic moving image 39.

Examples of the second display region 38 include assistance information 44 that assists the doctor 12 in medical determination or the like in the endoscopy. Examples of the assistance information 44 include various types of information about the subject 26 in which the endoscope 16 is inserted into the body, and/or various types of information obtained by performing a medical support process described below. In the example shown in FIG. 1, a reference image 44A is displayed in the second display region 38. The reference image 44A is an image referred to by the doctor 12 and is obtained by performing the medical support process described below.

The endoscopy of the large intestine 28 includes an insertion step of inserting the endoscope 16 into the large intestine 28 and a removal step of removing the endoscope 16 from the large intestine 28. In the insertion step, the endoscope 16 is inserted into the large intestine 28 along an insertion path 29A. For example, the insertion path 29A refers to a path from an anus (not shown) to an ileocecal portion 28A. In the removal step, the endoscope 16 is removed from the large intestine 28 along a removal path 29B. For example, the removal path 29B refers to a path from the ileocecal portion 28A to the anus. In the insertion step, in a case where the endoscope 16 reaches the ileocecal portion 28A of the large intestine 28 along the insertion path 29A, the process is switched from the insertion step to the removal step, and the endoscope 16 is removed from the large intestine 28 along the removal path 29B. In the insertion step, imaging of the inside of the large intestine 28 is performed by the endoscope apparatus 10. In the removal step, imaging of the inside of the large intestine 28 is performed by the endoscope apparatus 10 in the same manner as in the insertion step. In addition, in the removal step, a medical treatment (for example, discrimination, marking, resection, and/or hemostasis) is performed by the endoscope apparatus 10.

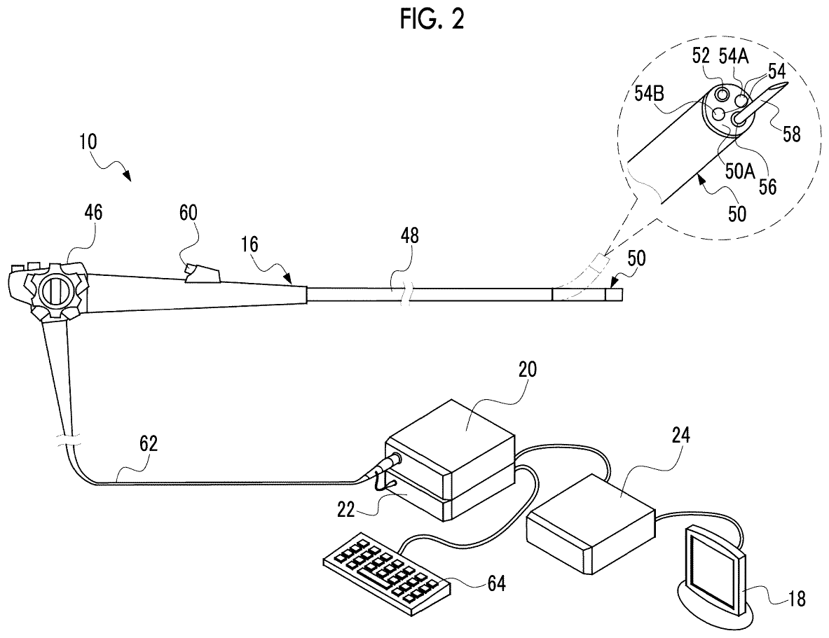

FIG. 2 is a conceptual diagram showing an example of an overall configuration of the endoscope apparatus 10. As shown in FIG. 2, the endoscope 16 comprises an operating part 46 and an insertion part 48. The insertion part 48 is partially bent by operating the operating part 46. The insertion part 48 is inserted into the large intestine 28 while being bent according to a shape of the large intestine 28 (see FIG. 1) in response to the operation of the operating part 46 by the doctor 12 (see FIG. 1).

A camera 52, an illumination device 54, and a treatment tool opening 56 are provided in a distal end part 50 of the insertion part 48. The camera 52 and the illumination device 54 are provided on a distal end surface 50A of the distal end part 50. Here, although a form example is described in which the camera 52 and the illumination device 54 are provided on the distal end surface 50A of the distal end part 50, this is merely an example. The camera 52 and the illumination device 54 may be provided on a side surface of the distal end part 50, so that the endoscope 16 may be configured as a side-viewing endoscope.

The camera 52 is inserted into a body cavity of the subject 26 to image the observation target region. In the first embodiment, the camera 52 acquires the endoscopic moving image 39 by imaging the inside of the body (for example, the inside of the large intestine 28) of the subject 26. Examples of the camera 52 include a CMOS camera. However, this is only an example, and the camera may be other types of camera such as a CCD camera.

The illumination device 54 has illumination windows 54A and 54B. The illumination device 54 emits the light 30 (see FIG. 1) through illumination windows 54A and 54B. Examples of the type of the light 30 emitted from the illumination device 54 include visible light (for example, white light) and invisible light (for example, near-infrared light). In addition, the illumination device 54 emits special light through the illumination windows 54A and 54B. Examples of the special light include light for BLI and/or light for LCI. The camera 52 images the inside of the large intestine 28 by an optical method in a state in which the light 30 is emitted inside the large intestine 28 by the illumination device 54.

The treatment tool opening 56 is an opening through which a treatment tool 58 protrudes from the distal end part 50. In addition, the treatment tool opening 56 is also used as a suction port for sucking blood, internal filth, and the like and a delivery port for sending out a fluid.

A treatment tool insertion port 60 is formed in the operating part 46, and the treatment tool 58 is inserted into the insertion part 48 through the treatment tool insertion port 60. The treatment tool 58 passes through the insertion part 48 and protrudes from the treatment tool opening 56 to the outside. In the example shown in FIG. 2, an aspect is shown in which a puncture needle protrudes from the treatment tool opening 56 as the treatment tool 58. Here, the puncture needle is illustrated as the treatment tool 58, but this is merely an example. The treatment tool 58 may be a grasping forceps, a papillotomy knife, a snare, a catheter, a guide wire, a cannula, and/or a puncture needle with a guide sheath, or the like.

The endoscope 16 is connected to the light source device 20 and the control device 22 via a universal cord 62. The medical support device 24 and a reception device 64 are connected to the control device 22. In addition, the display device 18 is connected to the medical support device 24. That is, the control device 22 is connected to the display device 18 via the medical support device 24.

Here, since the medical support device 24 is illustrated as an externally connected device for expanding a function performed by the control device 22, a form example is described in which the control device 22 and the display device 18 are indirectly connected to each other via the medical support device 24, but this is merely an example. For example, the display device 18 may be directly connected to the control device 22. In this case, for example, the functions of the medical support device 24 may be provided in the control device 22, or the control device 22 may be provided with a function of causing a server (not shown) to execute the same process as the process (for example, a medical support process which will be described below) executed by the medical support device 24, receiving a processing result of the server, and using the processing result.

The reception device 64 receives an instruction from the doctor 12 and outputs the received instruction as an electric signal to the control device 22. Examples of the reception device 64 include a keyboard, a mouse, a touch panel, a foot switch, a microphone, and/or a remote control device.

The control device 22 controls the light source device 20, transmits and receives various signals to and from the camera 52, or transmits and receives various signals to and from the medical support device 24.

The light source device 20 emits light under the control of the control device 22 and supplies the light to the illumination device 54. A light guide is provided in the illumination device 54, and the light supplied from the light source device 20 is emitted from the illumination windows 54A and 54B through the light guide. The control device 22 causes the camera 52 to perform imaging, acquires the endoscopic moving image 39 (see FIG. 1) from the camera 52, and outputs the endoscopic moving image 39 to a predetermined output destination (for example, the medical support device 24).

The medical support device 24 supports medical care (here, as an example, an endoscopy) by performing various types of image processing on the endoscopic moving image 39 input from the control device 22. The medical support device 24 outputs the endoscopic moving image 39 that has been subjected to various types of image processing to a predetermined output destination (for example, the display device 18).

Here, a form example is described in which the endoscopic moving image 39 output from the control device 22 is output to the display device 18 via the medical support device 24, but this is merely an example. For example, the control device 22 and the display device 18 may be connected to each other, and the endoscopic moving image 39 that has been subjected to the image processing by the medical support device 24 may be displayed on the display device 18 via the control device 22.

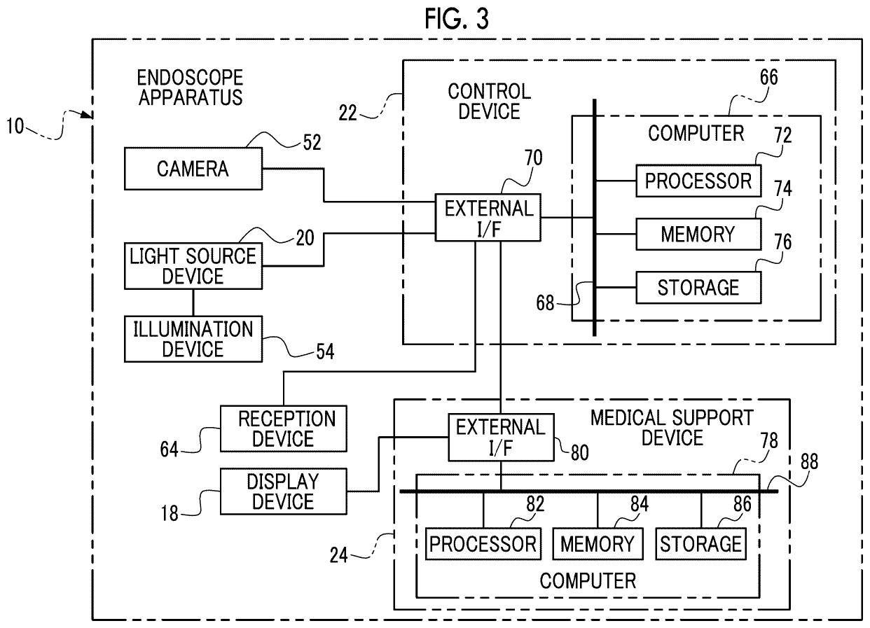

FIG. 3 is a block diagram showing an example of a hardware configuration of an electric system of the endoscope apparatus 10. As shown in FIG. 3, the control device 22 comprises a computer 66, a bus 68, and an external I/F 70. The computer 66 comprises a processor 72, a memory 74, and a storage 76. The processor 72, the memory 74, the storage 76, and the external I/F 70 are connected to the bus 68. The processor 72 controls the entire control device 22. The memory 74 and the storage 76 are used by the processor 72.

The external I/F 70 transmits and receives various types of information between one or more devices (hereinafter, also referred to as “first external devices”) outside the control device 22 and the processor 72.

As one of the first external devices, the camera 52 is connected to the external I/F 70, and the external I/F 70 transmits and receives various types of information between the camera 52 and the processor 72. The processor 72 controls the camera 52 via the external I/F 70. In addition, the processor 72 acquires the endoscopic moving image 39 (see FIG. 1) obtained by imaging the inside of the large intestine 28 (see FIG. 1) by the camera 52 via the external I/F 70.

As one of the first external devices, the light source device 20 is connected to the external I/F 70, and the external I/F 70 transmits and receives various types of information between the light source device 20 and the processor 72. The light source device 20 supplies light to the illumination device 54 under the control of the processor 72. The illumination device 54 performs irradiation with the light supplied from the light source device 20.

As one of the first external devices, the reception device 64 is connected to the external I/F 70. The processor 72 acquires the instruction received by the reception device 64 via the external I/F 70 and performs a process corresponding to the acquired instruction.

The medical support device 24 comprises a computer 78 and an external I/F 80. The computer 78 comprises a processor 82, a memory 84, and a storage 86. The processor 82, the memory 84, the storage 86, and the external I/F 80 are connected to a bus 88. In the first embodiment, the medical support device 24 is an example of a “medical support device” according to the present disclosure, the computer 78 is an example of a “computer” according to the present disclosure, and the processor 82 is an example of a “processor” according to the present disclosure.

Since a hardware configuration (that is, the processor 82, the memory 84, and the storage 86) of the computer 78 is basically the same as the hardware configuration of the computer 66, the hardware configuration of the computer 78 will not be described here.

The external I/F 80 transmits and receives various types of information between one or more devices (hereinafter, also referred to as “second external devices”) outside the medical support device 24 and the processor 82.

As one of the second external devices, the control device 22 is connected to the external I/F 80. In the example shown in FIG. 3, the external I/F 70 of the control device 22 is connected to the external I/F 80. The external I/F 80 transmits and receives various types of information between the processor 82 of the medical support device 24 and the processor 72 of the control device 22. For example, the processor 82 acquires the endoscopic moving image 39 (see FIG. 1) from the processor 72 of the control device 22 via the external I/Fs 70 and 80 and performs various types of image processing on the acquired endoscopic moving image 39.

As one of the second external devices, the display device 18 is connected to the external I/F 80. The processor 82 controls the display device 18 via the external I/F 80 so that various types of information (for example, the endoscopic moving image 39 subjected to various types of image processing) are displayed on the display device 18.

Meanwhile, in the endoscopy, the doctor 12 determines whether or not a medical treatment is necessary for the lesion 42 shown in the endoscopic moving image 39 while confirming the endoscopic moving image 39 via the display device 18, and performs the medical treatment on the lesion 42 in a case where the medical treatment is necessary.

In a general endoscopy, in a case where the doctor 12 finds the lesion 42 via the screen 35 (see FIG. 1) in the insertion step (see FIG. 1), the doctor 12 performs a medical treatment on the lesion 42 found in the insertion step, in the removal step. In this case, the doctor 12 needs to visually specify the lesion 42 found in the insertion step thoroughly in the removal step again.

For example, in a case where the number of the lesions 42 found in the insertion step is one, the doctor 12 can almost always find the lesion 42 found in the insertion step, even in the removal step. However, in a case where the number of the lesions 42 found in the insertion step is a plurality, the doctor 12 may overlook some of the plurality of lesions 42 found in the insertion step, in the removal step. The more the number of the lesions 42 found in the insertion step, the higher the possibility that the doctor 12 may overlook the lesions 42 found in the insertion step, in the removal step.

In order to ensure that the medical treatment is thoroughly performed on each of all the lesions 42 found in the endoscopy, it is very important that the lesion 42 found in the insertion step is visually specified thoroughly in the removal step again, regardless of the number of the lesions 42 found in the insertion step.

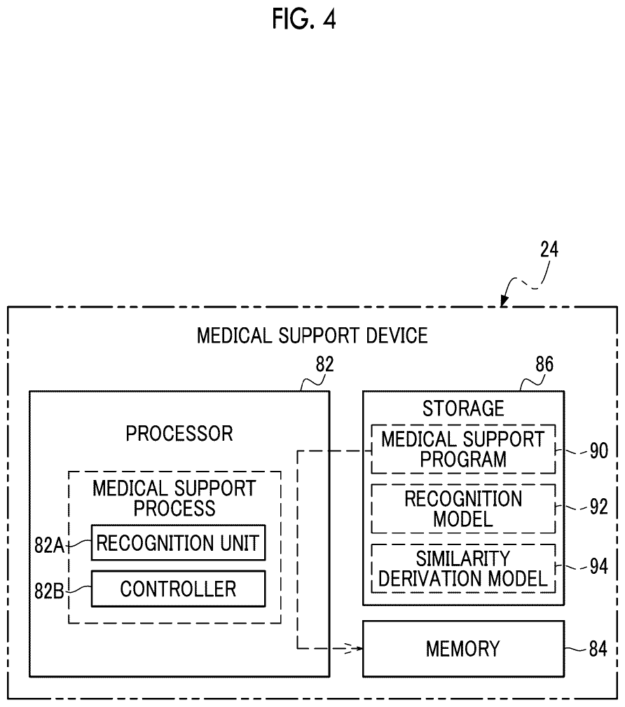

Therefore, in view of such circumstances, in the first embodiment, as shown in FIG. 4 as an example, the medical support process is performed by the processor 82 of the medical support device 24. FIG. 4 is a block diagram showing an example of functions of main units of the processor 82 included in the medical support device 24 and an example of information stored in the storage 86.

A medical support program 90 is stored in the storage 86. The medical support program 90 is an example of a “program” according to the present disclosure. The processor 82 reads out the medical support program 90 from the storage 86 and executes the read-out medical support program 90 on the memory 84 to perform the medical support process. The medical support process is realized by the processor 82 operating as a recognition unit 82A and a controller 82B according to the medical support program 90 executed on the memory 84.

The storage 86 stores a recognition model 92 and a similarity derivation model 94. Although the details will be described below, the recognition model 92 is used by the recognition unit 82A, and the similarity derivation model 94 is used by the controller 82B.

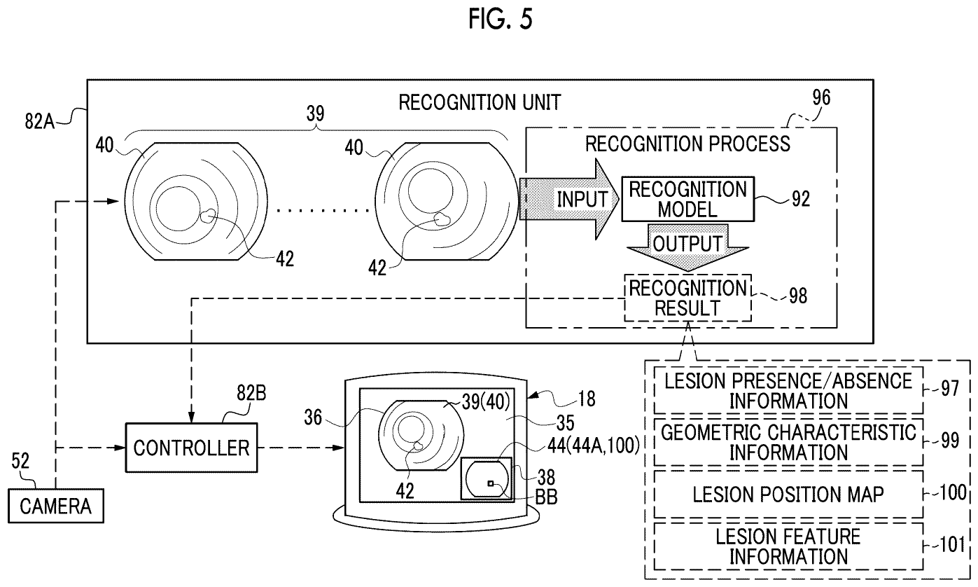

FIG. 5 is a conceptual diagram showing an example of processing contents of the recognition unit 82A and the controller 82B. As shown in FIG. 5, the recognition unit 82A and the controller 82B acquire each of the plurality of frames 40 along the time series included in the endoscopic moving image 39, which is generated by being captured by the camera 52 at an imaging frame rate (for example, several tens of frames/second), from the camera 52 in units of one frame along the time series.

The controller 82B outputs the endoscopic moving image 39 to the display device 18. For example, the controller 82B displays the endoscopic moving image 39 in the first display region 36 as a live view image. That is, each time the frame 40 is acquired from the camera 52, the controller 82B displays the acquired frame 40 in the first display region 36 in order at a display frame rate (for example, several tens of frames/second). In addition, the controller 82B displays the assistance information 44 in the second display region 38. In addition, for example, the controller 82B updates the display content (for example, the assistance information 44) of the second display region 38 in accordance with the display content of the first display region 36.

The recognition unit 82A recognizes the lesion 42 in the endoscopic moving image 39 by using the endoscopic moving image 39 acquired from the camera 52. That is, the recognition unit 82A recognizes the lesion 42 shown in the frame 40 by sequentially performing a recognition process 96 on each of the plurality of frames 40 along the time series included in the endoscopic moving image 39 acquired from the camera 52. For example, the recognition unit 82A recognizes geometric characteristics (for example, a position and a shape) of the lesion 42, a kind of the lesion 42, a type of the lesion 42 (for example, a pedunculated type, a subpedunculated type, a sessile type, a surface raised type, a surface flat type, a surface depressed type, and the like), and the like. In the first embodiment, the recognition process 96 is an example of an “object recognition process” according to the present disclosure.

The recognition process 96 is performed on the acquired frame 40 each time the frame 40 is acquired by the recognition unit 82A. The recognition process 96 is a process of recognizing the lesion 42 by a method using AI. Here, as the recognition process 96, a process using the recognition model 92 is performed. The recognition model 92 is a trained model for object recognition in a bounding box method using AI. Here, the recognition model 92 is an example of “object recognition AI” according to the present disclosure.

The recognition model 92 is optimized by performing machine learning on a neural network using first training data. The first training data is a data set including a plurality of data (that is, a plurality of frames of data) in which first example data and first correct answer data are associated with each other.

The first example data is an image assuming the frame 40. First examples of the image assuming the frame 40 includes an image obtained by actually imaging the inside of the large intestine with the camera. Second examples of the image assuming the frame 40 include an image virtually created. The first correct answer data is correct answer data (that is, an annotation) for the first example data. Here, as an example of the first correct answer data, an annotation for specifying geometric characteristics of a lesion shown in an image used as the first example data, a kind of the lesion, and a type of the lesion is used.

The recognition unit 82A acquires the frame 40 from the camera 52 and inputs the acquired frame 40 to the recognition model 92. As a result, the recognition model 92 recognizes the lesion 42 shown in the input frame 40 each time the frame 40 is input, and outputs a recognition result 98.

The recognition result 98 includes lesion presence/absence information 97. The lesion presence/absence information 97 is information indicating whether or not the lesion 42 is shown in the frame 40 input to the recognition model 92. In addition, in a case where the lesion 42 is shown in the frame 40 input to the recognition model 92, the recognition result 98 includes geometric characteristic information 99, a lesion position map 100, lesion feature information 101, and the like.

The geometric characteristic information 99 is information (for example, coordinates) for specifying a shape and a position of the lesion 42 in the frame 40. The lesion position map 100 is a map for specifying the position of the lesion 42 in the frame 40. The lesion feature information 101 is information for specifying the kind of the lesion 42 shown in the frame 40 input to the recognition model 92 and the type of the lesion 42.

Geometric characteristics (for example, a shape and a size of an outer contour) of the lesion position map 100 correspond to geometric characteristics (for example, a shape and a size of an outer contour) of the frame 40. The lesion position map 100 includes a bounding box BB. The bounding box BB is a rectangular frame (for example, a rectangular border circumscribing an image region showing the lesion 42) for specifying a position recognized by the recognition model 92 as the position of the lesion 42 in the frame 40, which is shown in the frame 40. The geometric characteristic information 99 is given to a geometric center of the bounding box BB (for example, a centroid of the bounding box BB).

In the example shown in FIG. 5, the lesion position map 100 is displayed on the screen 35 (for example, the second display region 38) by the controller 82B as the reference image 44A. In this case, the lesion position map 100 displayed on the screen 35 is updated according to a display frame rate applied to the first display region 36. That is, the display of the lesion position map 100 (that is, the display of the bounding box BB) in the second display region 38 is updated in synchronization with a display timing of the endoscopic moving image 39 displayed in the first display region 36. With such a configuration, the doctor 12 can ascertain an approximate position of the lesion 42 in the endoscopic moving image 39 displayed in the first display region 36 by referring to the lesion position map 100 displayed in the second display region 38 while observing the endoscopic moving image 39 displayed in the first display region 36.

The controller 82B may display the lesion feature information 101 on the screen 35 (for example, the second display region 38). In addition, the controller 82B may display the bounding box BB or an identifier (for example, a mark) instead of the bounding box BB in the first display region 36. In this case, for example, the controller 82B need only specify the shape and the position of the lesion 42 in the frame 40 from the geometric characteristic information 99, and display the bounding box BB or the identifier instead of the bounding box BB at the specified shape and position.

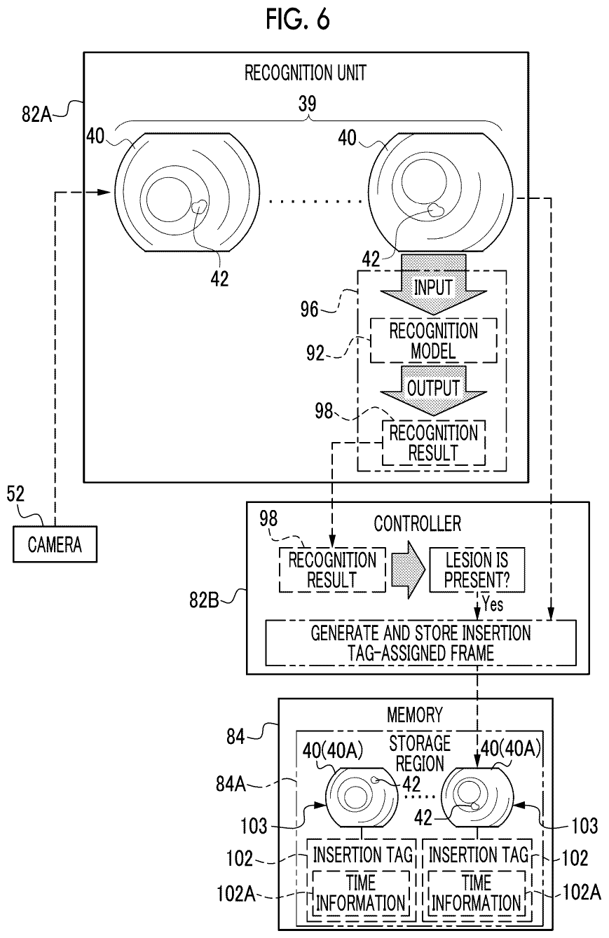

FIG. 6 is a conceptual diagram showing an example of processing contents of the recognition unit 82A and the controller 82B in the insertion step. As shown in FIG. 6, in the insertion step, the controller 82B acquires the recognition result 98 from the recognition unit 82A, and determines whether or not the lesion 42 is shown in the frame 40 input to the recognition model 92 with reference to the acquired recognition result 98 (for example, the lesion presence/absence information 97).

Here, in a case where the lesion 42 is shown in the frame 40 input to the recognition model 92 in order to obtain the recognition result 98 acquired from the recognition unit 82A by the controller 82B, the controller 82B generates an insertion tag-assigned frame 103.

The insertion tag-assigned frame 103 is a frame in which an insertion tag 102 is assigned to an insertion step frame 40A. In other words, the insertion tag-assigned frame 103 is a frame in which the insertion step frame 40A and the insertion tag 102 are associated with each other. The insertion step frame 40A is the frame 40 that is input to the recognition model 92 in order to obtain the recognition result 98 in the insertion step and in which the lesion 42 is shown. The insertion tag 102 is a tag for specifying that the insertion step frame 40A is the frame 40 obtained by imaging with the camera 52 in the insertion step. The insertion tag 102 includes time information 102A. The time information 102A is information indicating a time at which the insertion step frame 40A is obtained in the insertion step (that is, a time at which imaging for obtaining the insertion step frame 40A is performed). In the first embodiment, the insertion step frame 40A is an example of a “first medical image” according to the present disclosure.

The memory 84 is provided with a storage region 84A. The controller 82B stores the generated insertion tag-assigned frame 103 in the storage region 84A in time series each time the insertion tag-assigned frame 103 is generated. The storage region 84A is an example of a “first storage region” and a “second storage region” according to the present disclosure.

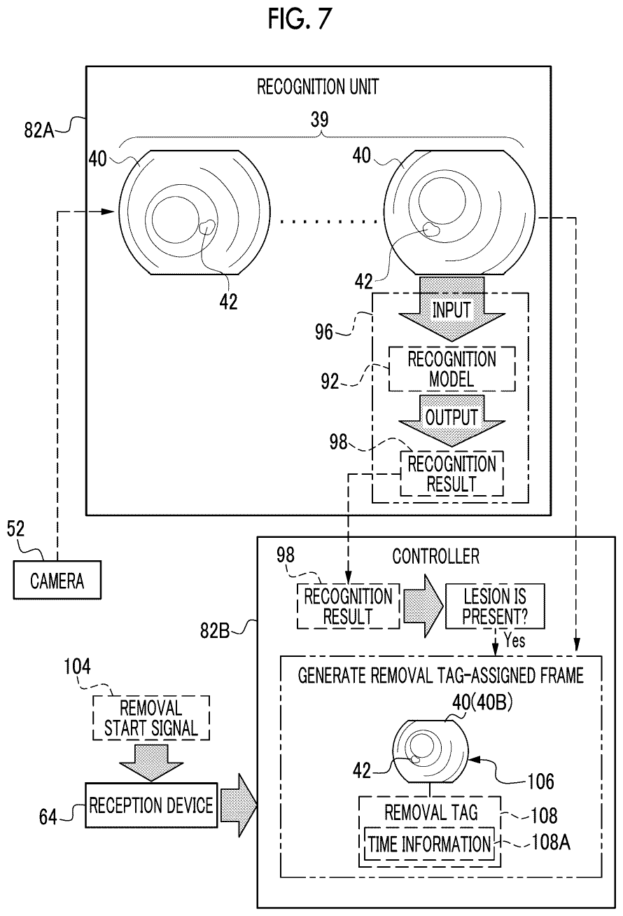

FIG. 7 is a conceptual diagram showing an example of processing contents of the recognition unit 82A and the controller 82B in the removal step. As shown in FIG. 7, at a timing at which the endoscopy enters the removal step, a removal start signal 104 is received by the reception device 64. The removal start signal 104 is a signal indicating that the removal step is started in the endoscopy. The controller 82B recognizes that the removal step is started in the endoscopy on a condition that the removal start signal 104 is received by the reception device 64. In the removal step, the controller 82B acquires the recognition result 98 from the recognition unit 82A, and determines whether or not the lesion 42 is shown in the frame 40 input to the recognition model 92 with reference to the acquired recognition result 98 (for example, the lesion presence/absence information 97).

Here, in a case where the lesion 42 is shown in the frame 40 input to the recognition model 92 in order to obtain the recognition result 98 acquired from the recognition unit 82A by the controller 82B, the controller 82B generates a removal tag-assigned frame 106.

The removal tag-assigned frame 106 is a frame in which a removal tag 108 is assigned to a removal step frame 40B. In other words, the removal tag-assigned frame 106 is a frame in which the removal step frame 40B and the removal tag 108 are associated with each other. The removal step frame 40B is the frame 40 obtained temporally later than the insertion step frame 40A. For example, the removal step frame 40B is the frame 40 that is input to the recognition model 92 in order to obtain the recognition result 98 in the removal step and in which the lesion 42 is shown. The removal tag 108 is a tag for specifying that the removal step frame 40B is the frame 40 obtained by imaging with the camera 52 in the removal step. The removal tag 108 includes time information 108A. The time information 108A is information indicating a time at which the removal step frame 40B is obtained in the removal step (that is, a time at which imaging for obtaining the removal step frame 40B is performed). In the first embodiment, the removal step frame 40B is an example of a “second medical image” according to the present disclosure.

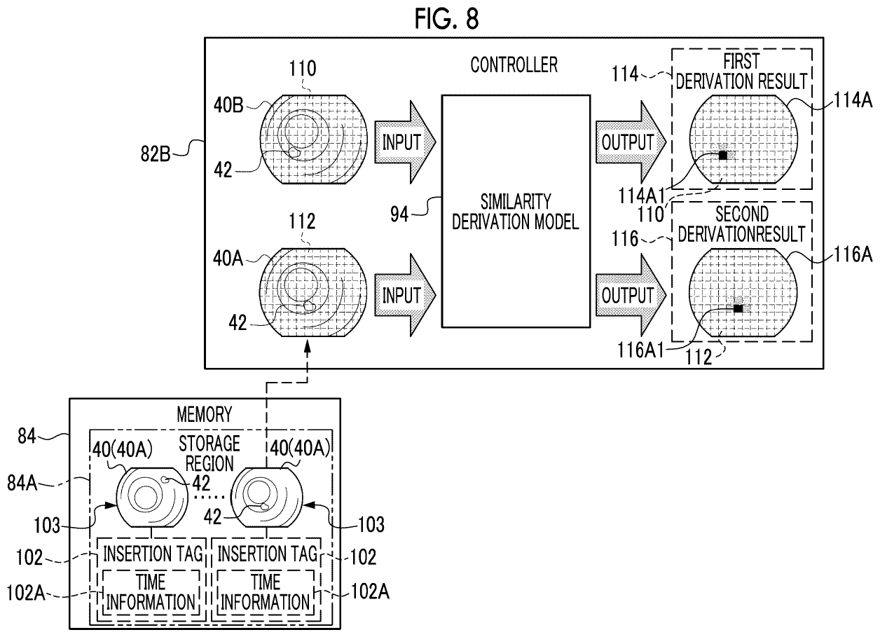

FIG. 8 is a conceptual diagram showing an example of processing contents of deriving a similarity between the lesion 42 shown in the insertion step frame 40A and the lesion 42 shown in the removal step frame 40B. As shown in FIG. 8, the controller 82B acquires the removal step frame 40B (that is, the removal step frame 40B used for generating the removal tag-assigned frame 106) and the insertion step frame 40A. Here, the insertion step frame 40A that is acquired by the controller 82B is an unprocessed insertion step frame 40A included in the insertion tag-assigned frame 103 stored in the storage region 84A. Here, the unprocessed insertion step frame 40A refers to the insertion step frame 40A that has not yet been input to the similarity derivation model 94 as the frame 40 paired with the removal step frame 40B shown in FIG. 8.

The removal step frame 40B has a plurality of divided regions 110. The plurality of divided regions 110 are obtained by dividing the removal step frame 40B according to a removal step frame division rule. Here, the removal step frame division rule refers to a rule for dividing the removal step frame 40B. Examples of the rule for dividing the removal step frame 40B include a rule in which a mesh formed in units of blocks (for example, vertical×horizontal=several pixels×several pixels) is applied to the removal step frame 40B to divide the removal step frame 40B into a mesh shape. In the first embodiment, the removal step frame division rule is an example of a “first rule” according to the present disclosure. In addition, the divided region 110 is an example of a “second divided region” according to the present disclosure.

The insertion step frame 40A includes a plurality of divided regions 112. The plurality of divided regions 112 are obtained by dividing the insertion step frame 40A according to an insertion step frame division rule. Here, the insertion step frame division rule refers to a rule for dividing the insertion step frame 40A. Examples of the rule for dividing the insertion step frame 40A include a rule in which a mesh formed in units of blocks (for example, vertical×horizontal=several pixels×several pixels) is applied to the insertion step frame 40A to divide the insertion step frame 40A into a mesh shape. In the first embodiment, the insertion step frame division rule is an example of a “first rule” according to the present disclosure. In addition, the divided region 112 is an example of a “first divided region” according to the present disclosure.

In the first embodiment, the removal step frame division rule and the insertion step frame division rule are the same rule. Therefore, shapes, sizes, and the number of the plurality of divided regions 110 and shapes, sizes, and the number of the plurality of divided regions 112 match. The removal step frame division rule and the insertion step frame division rule may be rules different from each other.

The similarity derivation model 94 is an example of “AI” according to the present disclosure. The similarity derivation model 94 is optimized by performing machine learning on the neural network using second training data. The second training data is a data set including a plurality of data (that is, a plurality of frames of data) in which second example data and second correct answer data are associated with each other.

The second example data is a frame set of a frame assuming the insertion step frame 40A (hereinafter, referred to as an “insertion step example frame”) and a frame assuming the removal step frame 40B (hereinafter, referred to as a “removal step example frame”). First examples of the insertion step example frame include an image in which an image obtained by actually imaging the inside of the large intestine with the camera is divided according to the insertion step frame division rule. Second examples of the insertion step example frame include an image that is virtually created and is divided according to the insertion step frame division rule. First examples of the removal step example frame include an image in which an image obtained by actually imaging the inside of the large intestine with the camera is divided according to the removal step frame division rule. Second examples of the removal step example frame include an image that is virtually created and is divided according to the removal step frame division rule.

The second correct answer data is correct answer data (that is, an annotation) for the second example data. The second correct answer data is associated with each of a plurality of divided regions obtained by dividing the insertion step example frame according to the insertion step frame division rule (hereinafter, referred to as “insertion step example divided region”), and each of a plurality of divided regions obtained by dividing the removal step example frame according to the removal step frame division rule (hereinafter, referred to as “removal step example divided region”). Examples of the second correct answer data include data indicating whether or not the lesion is shown in the insertion step example divided region and the removal step example divided region and indicating whether or not the lesion shown in the insertion step example divided region and the lesion shown in the removal step example divided region in a case where the lesions are shown in the insertion step example divided region and in the removal step example divided region are the same.

The controller 82B inputs the insertion step frame 40A and the removal step frame 40B to the similarity derivation model 94. As a result, the similarity derivation model 94 derives a similarity which is a degree to which the lesion 42 shown in the divided region 110 and the lesion 42 shown in the divided region 112 are similar to each other in a case where the lesions 42 are shown in the divided region 110 and the divided region 112 that correspond to each other in position. The similarity is derived for each of the divided regions 110 for the removal step frame 40B and is derived for each of the divided regions 112 for the insertion step frame 40A. Then, the similarity derivation model 94 outputs a first derivation result 114 corresponding to the removal step frame 40B and a second derivation result 116 corresponding to the insertion step frame 40A each time a pair of the removal step frame 40B and the insertion step frame 40A is input.

The first derivation result 114 includes a similarity for each of the plurality of divided regions 110 and a map 114A showing a distribution of similarities. The map 114A is an example of a “map” according to the present disclosure. In the first embodiment, as the map 114A, a feature amount map in which a distribution of similarities exceeding a threshold value is represented in shading according to the similarity is used. Here, the similarity is defined as 0% to 100%, and 85% is used as the threshold value in this case. In the example shown in FIG. 8, a high similarity distribution region 114A1 is shown as a region in which the similarities exceeding the threshold value are distributed in the map 114A.

The second derivation result 116 includes a similarity for each of the plurality of divided regions 112 and a map 116A showing a distribution of similarities. The map 116A is an example of a “map” according to the present disclosure. In the first embodiment, as the map 116A, a feature amount map in which a distribution of similarities exceeding a threshold value is represented in shading according to the similarity is used. Here, the similarity is defined as 0% to 100%, and 85% is used as the threshold value in this case. In the example shown in FIG. 8, a high similarity distribution region 116A1 is shown as a region in which the similarities exceeding the threshold value are distributed in the map 116A.

The controller 82B performs the process shown in FIG. 8 until the high similarity distribution region 114A1 and high similarity distribution region 116A1 are obtained, or until derivation of a similarity between one removal step frame 40B and each of all the insertion step frames 40A stored in the storage region 84A shown in FIG. 8 (that is, derivation of a similarity using the similarity derivation model 94) is completed. The process shown in FIG. 8 refers to inference using the similarity derivation model 94 for the pair of the insertion step frame 40A and the removal step frame 40B. The inference using the similarity derivation model 94 for the pair of the insertion step frames 40A and the removal step frames 40B refers to processing in which the pair of the insertion step frame 40A and the removal step frame 40B is input to the similarity derivation model 94 to derive the similarity by the similarity derivation model 94, and the first derivation result 114 and the second derivation result 116 are output.

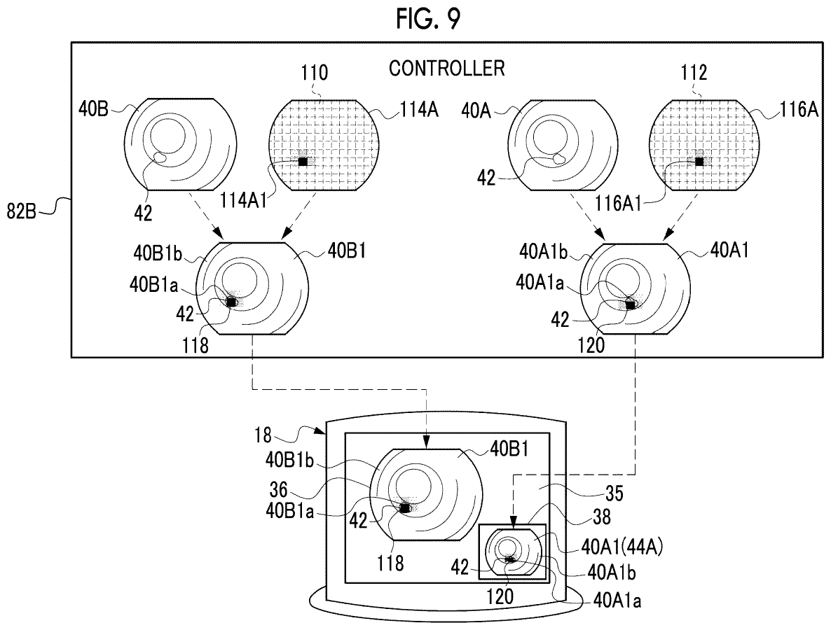

FIG. 9 is a conceptual diagram showing an example of processing contents using the removal step frame 40B and the map 114A by the controller 82B and processing contents using the insertion step frame 40A and the map 116A by the controller 82B in a case where the high similarity distribution regions 114A1 and 116A1 are obtained. As shown in FIG. 9, in a case where the map 114A including the high similarity distribution region 114A1 and the map 116A including the high similarity distribution region 116A1 are obtained from the similarity derivation model 94 (see FIG. 8), the controller 82B generates a removal step combined frame 40B1 based on the removal step frame 40B and the map 114A. Then, the controller 82B executes a removal step combined frame display process. The removal step combined frame display process is an example of a “second output process” according to the present disclosure. The removal step combined frame display process is a process of displaying the removal step combined frame 40B1 in the first display region 36. By performing the removal step combined frame display process, the removal step combined frame 40B1 is displayed in the first display region 36 in the live view method.

The removal step combined frame 40B1 is a frame in which the removal step frame 40B and a first lesion specifying image 118 corresponding to the high similarity distribution region 114A1 are combined. In the example shown in FIG. 9, a frame in which the first lesion specifying image 118 is superimposed on the removal step frame 40B is shown as the removal step combined frame 40B1.

The first lesion specifying image 118 is superimposed on a region, which is at a position corresponding to a position of the high similarity distribution region 114A1 in the map 114A, in the entire region of the removal step frame 40B. Here, as an example of the first lesion specifying image 118, the high similarity distribution region 114A1 is adopted as it is. However, this is merely an example, and an image in which the high similarity distribution region 114A1 is processed may be used. For example, an image (for example, a mark or the like) obtained by adjusting a color, a density, and/or a brightness of the high similarity distribution region 114A1 may be used. In addition, a transmittance of the first lesion specifying image 118 may be adjusted by alpha blending. Examples of the transmittance of the first lesion specifying image 118 include a transmittance to the extent that the doctor 12 can visually perceive the first lesion specifying image 118 and is not visually obstructed in observing the lesion 42 via the first display region 36 or performing a medical treatment on the lesion 42.

As described above, the removal step combined frame 40B1 obtained by superimposing the first lesion specifying image 118 on the removal step frame 40B is represented in an aspect in which a first image region 40B1a, which is an image region in which the lesion 42 is shown in the removal step frame 40B and a second image region 40B1b, which is an image region in which a portion other than the lesion 42 is shown in the removal step frame 40B, are discriminable from each other by the first lesion specifying image 118. In the example shown in FIG. 9, in the removal step combined frame 40B1, the first image region 40B1a is emphasized more than the second image region 40B1b by the first lesion specifying image 118. Here, the image region in which the lesion 42 is shown in the removal step frame 40B, that is, the first image region 40B1a which is a part of the removal step frame 40B is determined in units of the divided region 110. In addition, the image region in which a portion other than the lesion 42 is shown in the removal step frame 40B, that is, the second image region 40B1b which is a part of the removal step frame 40B is also determined in units of the divided region 110.

In the first embodiment, the first image region 40B1a is an example of a “second partial region” according to the present disclosure, and the second image region 40B1b is an example of a “second other region” according to the present disclosure.

In a case where the map 114A including the high similarity distribution region 114A1 and the map 116A including the high similarity distribution region 116A1 are obtained from the similarity derivation model 94 (see FIG. 8), the controller 82B generates an insertion step combined frame 40A1 based on the insertion step frame 40A and the map 116A. Then, the controller 82B executes an insertion step combined frame display process. The insertion step combined frame display process is an example of a “first output process” according to the present disclosure. The insertion step combined frame display process is a process of displaying the insertion step combined frame 40A1 in the second display region 38 as one of the reference images 44A.

The insertion step combined frame 40A1 is a frame in which the insertion step frame 40A and a second lesion specifying image 120 corresponding to the high similarity distribution region 116A1 are combined. In the example shown in FIG. 9, a frame in which the second lesion specifying image 120 is superimposed on the insertion step frame 40A is shown as the insertion step combined frame 40A1.

The second lesion specifying image 120 is superimposed on a region, which is at a position corresponding to a position of the high similarity distribution region 116A1 in the map 116A, in the entire region of the insertion step frame 40A. Here, as an example of the second lesion specifying image 120, the high similarity distribution region 116A1 is adopted as it is. However, this is merely an example, and an image in which the high similarity distribution region 116A1 is processed may be used. For example, an image (for example, a mark or the like) obtained by adjusting a color, a density, and/or a brightness of the high similarity distribution region 116A1 may be used. In addition, a transmittance of the second lesion specifying image 120 may be adjusted by alpha blending. Examples of the transmittance of the second lesion specifying image 120 include a transmittance to the extent that the doctor 12 can visually perceive the second lesion specifying image 120 and is not visually obstructed in observing the lesion 42 via the second display region 38 or performing a medical treatment on the lesion 42.

As described above, the insertion step combined frame 40A1 obtained by superimposing the second lesion specifying image 120 on the insertion step frame 40A is represented in an aspect in which a third image region 40A1a, which is an image region in which the lesion 42 is shown in the insertion step frame 40A and a fourth image region 40A1b, which is an image region in which a portion other than the lesion 42 is shown in the insertion step frame 40A, are discriminable from each other by the second lesion specifying image 120. In the example shown in FIG. 9, in the insertion step combined frame 40A1, the third image region 40A1a is emphasized more than the fourth image region 40A1b by the second lesion specifying image 120. Here, the image region in which the lesion 42 is shown in the insertion step frame 40A, that is, the third image region 40A1a which is a part of the insertion step frame 40A is determined in units of the divided region 112. In addition, the image region in which a portion other than the lesion 42 is shown in the insertion step frame 40A, that is, the fourth image region 40A1b which is a part of the insertion step frame 40A is also determined in units of the divided region 112.

In the first embodiment, the third image region 40A1a is an example of a “first partial region” according to the present disclosure, and the fourth image region 40A1b is an example of a “first other region” according to the present disclosure.

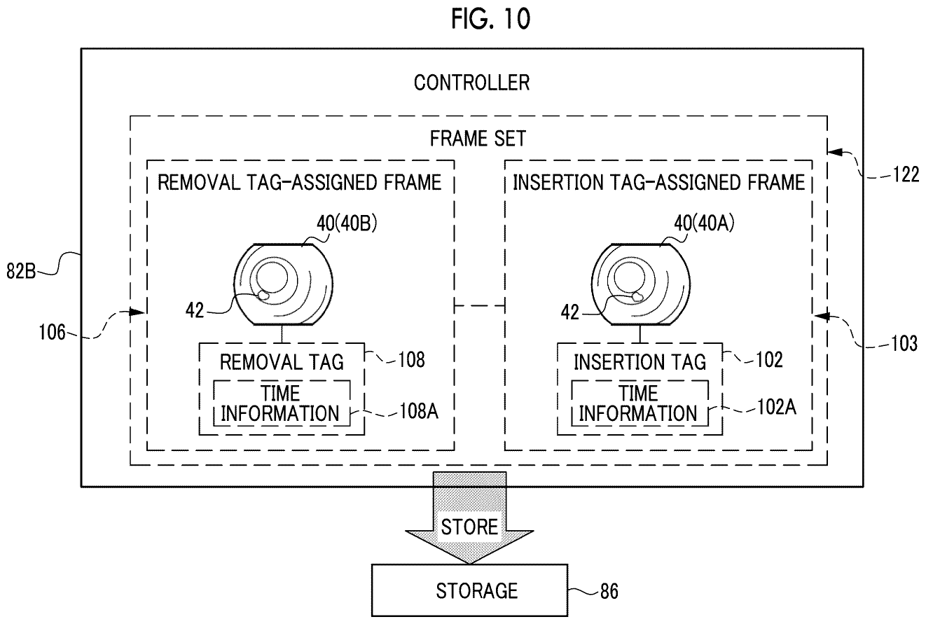

FIG. 10 is a conceptual diagram showing an example of processing contents using the insertion tag-assigned frame 103 and the removal tag-assigned frame 106 by the controller 82B. As shown in FIG. 10, the controller 82B generates a frame set 122 in which the insertion tag-assigned frame 103 and the removal tag-assigned frame 106, which includes the removal step frame 40B in which the same lesion 42 as the lesion 42 shown in the insertion step frame 40A included in the insertion tag-assigned frame 103 is shown, are associated with each other. The controller 82B stores the generated frame set 122 in the storage 86. The controller 82B stores the generated frame set 122 in the storage 86 in time series each time the frame set 122 is generated.

The insertion tag-assigned frame 103 used for generating the frame set 122 by the controller 82B is the insertion tag-assigned frame 103 including the removal step frame 40B input to the similarity derivation model 94 in order to obtain the first derivation result 114 including the high similarity distribution region 114A1 among all the insertion tag-assigned frames 103 stored in the storage region 84A. The removal tag-assigned frame 106 used for generating the frame set 122 by the controller 82B is the removal tag-assigned frame 106 including the removal step frame 40B input to the similarity derivation model 94 in a pair with the insertion step frame 40A in which the same lesion 42 is shown, in order to obtain the second derivation result 116 including the high similarity distribution region 116A1.

That is, the frame set 122 is a pair of the removal tag-assigned frame 106 including the removal step frame 40B used in the removal step combined frame 40B1 shown in FIG. 9 and the insertion tag-assigned frame 103 including the insertion step frame 40A used in the insertion step combined frame 40A1 shown in FIG. 9.

In the example shown in FIG. 10, a form example is described in which the frame set 122 is stored in the storage 86, but this is merely an example. The frame set 122 may be stored in a storage region other than the storage 86. Examples of the storage region other than the storage 86 include an electronic medical record, a server, and/or a tablet terminal.

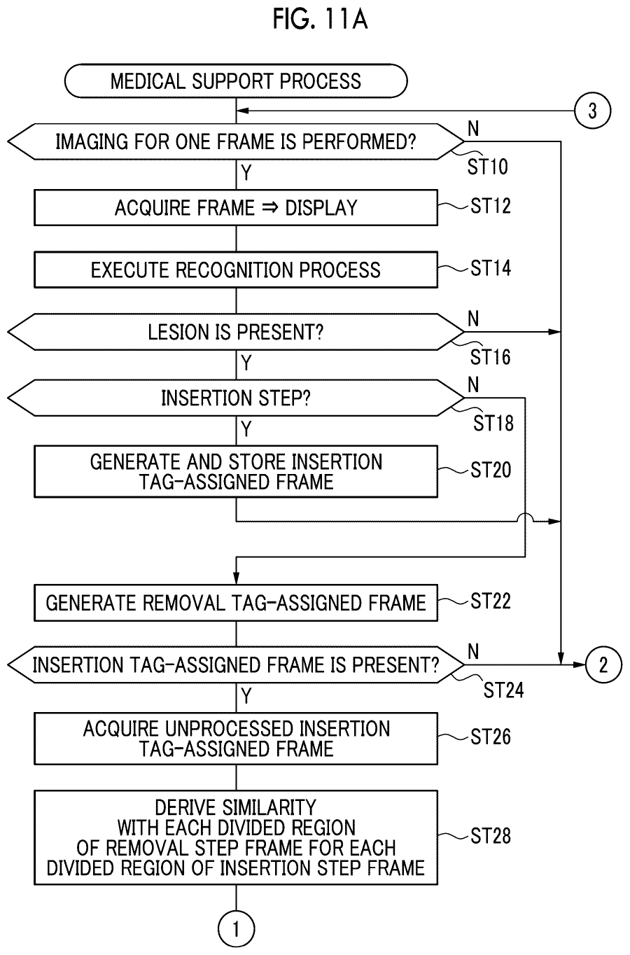

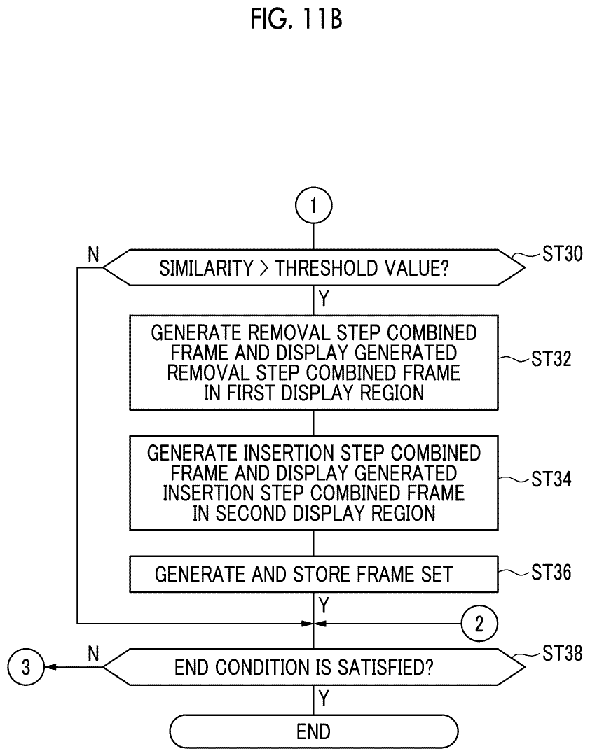

Next, an operation of a part of the endoscope apparatus 10 according to the present disclosure will be described with reference to FIGS. 11A and 11B. A flow of a medical support process shown in FIGS. 11A and 11B is an example of a “medical support method” according to the present disclosure.

In the following description, it is assumed that the inside of the large intestine 28 is irradiated with the same type of light (for example, light for BLI and/or light for LCI) in the insertion step and the removal step, and a region (for example, the intestinal wall 32) irradiated with the light is imaged by the camera 52.

In the medical support process shown in FIG. 11A, first, in step ST10, the recognition unit 82A determines whether or not imaging for one frame is performed by the camera 52 in the large intestine 28. In step ST10, in a case where the imaging for one frame is not performed by the camera 52 in the large intestine 28, a negative determination is made, and the medical support process proceeds to step ST38 shown in FIG. 11B. In step ST10, in a case where the imaging for one frame is performed by the camera 52 in the large intestine 28, a positive determination is made, and the medical support process proceeds to step ST12.

In step ST12, the recognition unit 82A and the controller 82B acquire the frame 40 obtained by imaging the large intestine 28 by the camera 52. Then, the controller 82B displays the frame 40 in the first display region 36. After the process in step ST12 is executed, the medical support process proceeds to step ST14.

In step ST14, the recognition unit 82A executes the recognition process 96 on the frame 40 acquired in step ST12. After the process in step ST14 is executed, the medical support process proceeds to step ST16.

In step ST16, the controller 82B determines whether or not the lesion 42 is shown in the frame 40 acquired in step ST12 (that is, whether or not the lesion 42 is recognized by the recognition unit 82A) based on the recognition result 98 obtained by performing the recognition process 96 in step ST14. In step ST16, in a case where the lesion 42 is not shown in the frame 40 acquired in step ST12, a negative determination is made, and the medical support process proceeds to step ST38 shown in FIG. 11B. In step ST16, in a case where the lesion 42 is shown in the frame 40 acquired in step ST12, a positive determination is made, and the medical support process proceeds to step ST18.

In step ST18, the controller 82B determines whether or not the endoscopy is the insertion step. In a case where the removal start signal 104 is not received by the reception device 64, it is determined that the endoscopy is the insertion step, and in a case where the removal start signal 104 is received by the reception device 64, it is determined that the endoscopy is not the insertion step (that is, the endoscopy is the removal step). In step ST18, in a case where the endoscopy is not the insertion step, a negative determination is made, and the medical support process proceeds to step ST22. In step ST18, in a case where the endoscopy is the insertion step, a positive determination is made, and the medical support process proceeds to step ST20.

In step ST20, the controller 82B generates the insertion tag-assigned frame 103 based on the frame 40 acquired in step ST12. Then, the controller 82B stores the generated insertion tag-assigned frame 103 in the storage region 84A. After the process in step ST20 is executed, the medical support process proceeds to step ST38 shown in FIG. 11B.

In step ST22, the controller 82B generates the removal tag-assigned frame 106 based on the frame 40 acquired in step ST12. After the process in step ST22 is executed, the medical support process proceeds to step ST24.

In step ST24, the controller 82B determines whether or not the insertion tag-assigned frame 103 is stored in the storage region 84A. In step ST24, in a case where the insertion tag-assigned frame 103 is not stored in the storage region 84A, a negative determination is made, and the medical support process proceeds to step ST38 shown in FIG. 11B. In step ST24, in a case where the insertion tag-assigned frame 103 is stored in the storage region 84A, a positive determination is made, and the medical support process proceeds to step ST26.

In step ST26, the controller 82B acquires the insertion tag-assigned frames 103 including the unprocessed insertion step frame 40A among all the insertion tag-assigned frames 103 stored in the storage region 84A. After the process in step ST26 is executed, the medical support process proceeds to step ST28.

In step ST28, the controller 82B derives a similarity with each divided region 110 of the removal step frame 40B included in the removal tag-assigned frame 106 generated in step ST22 for each divided region 112 of the insertion step frame 40A included in the insertion tag-assigned frame 103 acquired in step ST26 by using the similarity derivation model 94 (see FIG. 8). The similarity derivation model 94 outputs the first derivation result 114 including the similarity for each divided region 110 and the map 114A, and the second derivation result 116 including the similarity for each divided region 112 and the map 116A (see FIG. 8). After the process in step ST28 is executed, the medical support process proceeds to step ST30 shown in FIG. 11B.

In step ST30 shown in FIG. 11B, the controller 82B determines whether or not the similarity derived in step ST28 exceeds a threshold value. In step ST30, in a case where the similarity derived in step ST28 does not exceed the threshold value, a negative determination is made, and the medical support process proceeds to step ST38. In step ST30, in a case where the similarity derived in step ST28 exceeds the threshold value, a positive determination is made, and the medical support process proceeds to step ST32.

In step ST32, the controller 82B generates the removal step combined frame 40B1 based on the map 114A obtained by executing the process in step ST28 and the removal step frame 40B included in the removal tag-assigned frame 106 generated in step ST22 (see FIG. 9). Then, the controller 82B displays the generated removal step combined frame 40B1 in the first display region 36 (see FIG. 9). After the process in step ST32 is executed, the medical support process proceeds to step ST34.

In step ST34, the controller 82B generates the insertion step combined frame 40A1 based on the map 116A obtained by executing the process in step ST28 and the insertion step frame 40A used in step ST28 (see FIG. 9). Then, the controller 82B displays the generated insertion step combined frame 40A1 in the second display region 38 (see FIG. 9). After the process in step ST34 is executed, the medical support process proceeds to step ST36.

In step ST36, the controller 82B generates the frame set 122 based on the removal step combined frame 40B1 generated in step ST32 and the insertion step combined frame 40A1 generated in step ST34 (see FIG. 10). Then, the controller 82B stores the generated frame set 122 in the storage 86 (see FIG. 10). After the process in step ST36 is executed, the medical support process proceeds to step ST38.

In step ST38, the controller 82B determines whether or not a medical support process end condition is satisfied. An example of the medical support process end condition is a condition that an instruction for the endoscope apparatus 10 to end the medical support process is given (for example, a condition that the reception device 64 receives an instruction to end the medical support process).

In a case where the medical support process end condition is not satisfied in step ST38, a negative determination is made, and the medical support process proceeds to step ST10. In a case where the medical support process end condition is satisfied in step ST38, a positive determination is made, and the medical support process ends.

As described above, in the endoscope apparatus 10, in a case where the similarity between the lesion 42 shown in the insertion step frame 40A and the lesion 42 shown in the removal step frame 40B exceeds the threshold value, the insertion step combined frame 40A1 is displayed in the second display region 38, and the removal step combined frame 40B1 is displayed in the first display region 36.

The insertion step combined frame 40A1 displayed in the second display region 38 is represented in an aspect in which the third image region 40A1a in which the lesion 42 is shown and the fourth image region 40A1b in which the lesion 42 is not shown are discriminable from each other. For example, the second lesion specifying image 120 is superimposed on the third image region 40A1a in which the lesion 42 is shown, so that the third image region 40A1a is emphasized more than the fourth image region 40A1b.

Meanwhile, the removal step combined frame 40B1 displayed in the first display region 36 is represented in an aspect in which the first image region 40B1a in which the lesion 42 is shown and the second image region 40B1b in which the lesion 42 is not imaged are discriminable from each other. For example, the first lesion specifying image 118 is superimposed on the first image region 40B1a in which the lesion 42 is shown, so that the first image region 40B1a is emphasized more than the second image region 40B1b.

Here, the lesion 42 shown in the third image region 40A1a is a lesion observed by the doctor 12 in the insertion step, and the lesion 42 shown in the first image region 40B1a is a lesion observed by the doctor 12 in the removal step. The first lesion specifying image 118 that is superimposed on the first image region 40B1a and the second lesion specifying image 120 that is superimposed on the third image region 40A1a are images that are not displayed in a case where the similarity does not exceed the threshold value.

Therefore, by displaying the second lesion specifying image 120 to be superimposed on the third image region 40A1a or by displaying the first lesion specifying image 118 to be superimposed on the first image region 40B1a, the doctor 12 can visually specify that the lesion 42 shown in the first image region 40B1a and the lesion 42 shown in the third image region 40A1a are the same lesion. As a result, it is possible to contribute to allowing the doctor 12 to thoroughly observe again, via the endoscope 16, the lesion 42 observed in the large intestine 28 via the endoscope 16 by the doctor 12 while the endoscope 16 is inserted into the large intestine 28. For example, it is possible to contribute to allowing the doctor 12 to thoroughly observe again in the removal step, via the endoscope 16, the lesion 42 observed in the large intestine 28 via the endoscope 16 by the doctor 12 in the insertion step.

In addition, in the endoscope apparatus 10, since the first image region 40B1a is emphasized more than the second image region 40B1b, or the third image region 40A1a is emphasized more than the fourth image region 40A1b, the doctor 12 can easily visually specify the position of the lesion 42 from the removal step combined frame 40B1 displayed in the first display region 36 and can easily visually specify the position of the lesion 42 from the insertion step combined frame 40A1 displayed in the second display region 38.

In addition, in the endoscope apparatus 10, the third image region 40A1a on which the second lesion specifying image 120 is superimposed is defined in units of the divided region 112, and the first image region 40B1a on which the first lesion specifying image 118 is superimposed is defined in units of the divided region 110. Therefore, the image region in which the similarity exceeds the threshold value in the insertion step frame 40A can be specified in units of the divided region 112, and the image region in which the similarity exceeds the threshold value in the removal step frame 40B can be specified in units of the divided region 110.

In addition, in the endoscope apparatus 10, the high similarity distribution region 114A1 in which the similarity exceeds the threshold value is extracted from the plurality of divided regions 110 according to the map 114A, and the high similarity distribution region 116A1 in which the similarity exceeds the threshold value is extracted from the plurality of divided regions 112 according to the map 116A. As a result, one or more divided regions 110 in which the similarity exceeds the threshold value can be accurately extracted as the high similarity distribution region 114A1, and one or more divided regions 112 in which the similarity exceeds the threshold value can be accurately extracted as the high similarity distribution region 116A1. The maps 114A and 116A are derived by an AI method by using the similarity derivation model 94. Therefore, the maps 114A and 116A can be accurately derived as compared to a case where the maps 114A and 116A are derived only based on intuition and experience of the doctor 12.

In addition, in the endoscope apparatus 10, the inside of the large intestine 28 is irradiated with the same type of light (for example, light for BLI and/or light for LCI) in the insertion step and the removal step, and a region (for example, the intestinal wall 32) irradiated with the light is imaged by the camera 52. Accordingly, it is possible to suppress an occurrence of a situation in which the similarity does not exceed the threshold value because the type of light used in the imaging for obtaining the insertion step frame 40A and the type of light used in the imaging for obtaining the removal step frame 40B are different from each other.

In addition, in the endoscope apparatus 10, the frame set 122 is stored in the storage 86. In the frame set 122, the insertion step frame 40A and the removal step frame 40B in which the same lesion 42 is shown are associated with each other. Therefore, the doctor 12 can determine whether or not the lesion 42, which is visually recognized in the insertion step, is also visually recognized thoroughly in the removal step by confirming the content of the frame set 122 stored in the storage 86.

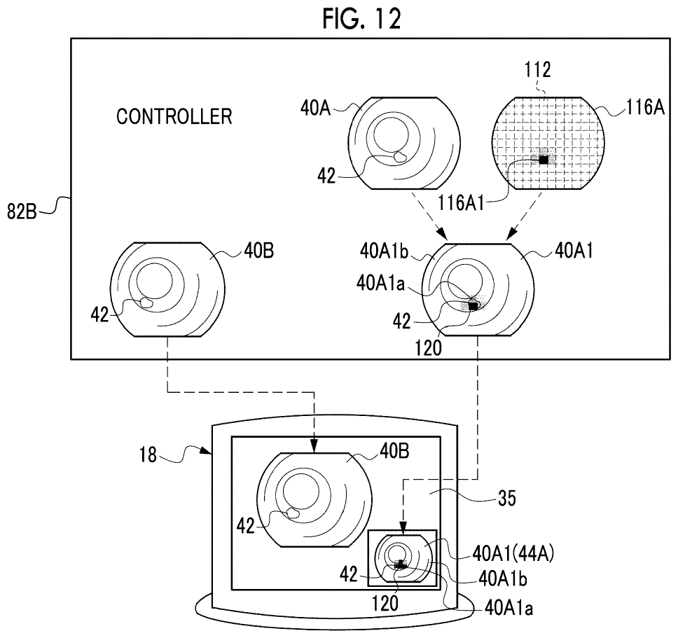

In the first embodiment, a form example is described in which the removal step combined frame 40B1 is displayed in the first display region 36, and the insertion step combined frame 40A1 is displayed in the second display region 38, but this is merely an example. For example, as shown in FIG. 12, the removal step frame 40B may be displayed in the first display region 36, and the insertion step combined frame 40A1 may be displayed in the second display region 38 in the same manner as in the first embodiment. In addition, in the same manner as in the first embodiment, the removal step combined frame 40B1 may be displayed in the first display region 36, and the insertion step frame 40A may be displayed in the second display region 38.

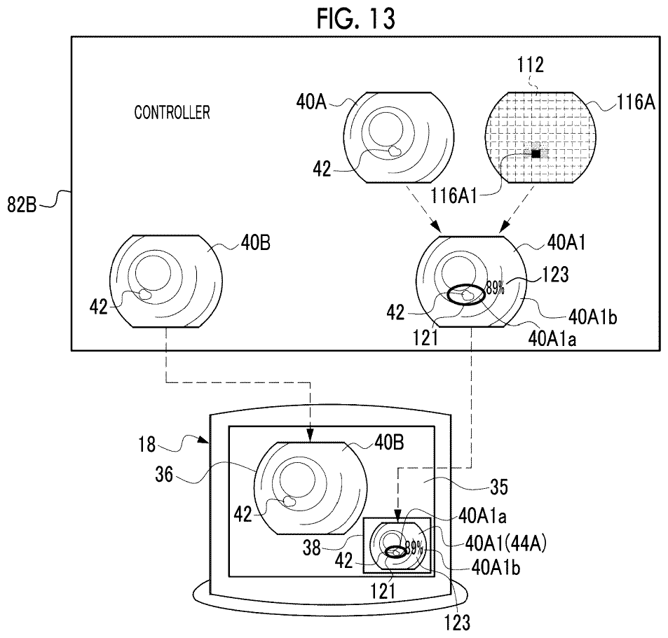

In the first embodiment, a form example is described in which the second lesion specifying image 120 is included in the insertion step combined frame 40A1, but this is merely an example. For example, as shown in FIG. 13, a border 121 may be used instead of the second lesion specifying image 120. The border 121 is a border that circumscribes the third image region 40A1a. In the example shown in FIG. 13, an elliptical border is illustrated as an example of the border 121, but this is merely an example. The border 121 need only be a border that can specify the third image region 40A1a (for example, a border along an outer edge of the third image region 40A1a), that is, a border that can discriminate between the third image region 40A1a and the fourth image region 40A1b. The border 121 may be an intermittent border in which a part of the border 121 is cut out, instead of a continuous line. In addition, as shown in FIG. 13 as an example, a numerical value 123 indicating a representative similarity may be displayed on the screen 35. Examples of the representative similarity include a maximum value of similarities of all the divided regions 112 included in the high similarity distribution region 116A1. The maximum value is merely an example, and a statistical value such as an average value, a minimum value, a median value, and/or a mode value may be displayed together with the maximum value or instead of the maximum value. A display position of the numerical value 123 may be a position adjacent to the border 121 in the second display region 38 or may be a position outside the second display region 38. The numerical value 123 need only be displayed in an aspect associated with the border 121 (for example, an aspect using a word balloon attached to the border 121). In addition, an identifier indicating a degree of similarity may be used instead of the numerical value 123.

In the example shown in FIG. 13, a form example is described in which the border 121 is used instead of the second lesion specifying image 120, but similarly, the border may be used instead of the first lesion specifying image 118. In this case as well, it is sufficient that the border can discriminate between the first image region 40B1a and the second image region 40B 1b. In addition, similarly to the numerical value 123 displayed in the second display region 38, a numerical value indicating a representative similarity may be displayed in the first display region 36. Examples of the representative numerical value displayed in the first display region 36 include a statistical value (for example, a maximum value, an average value, a minimum value, a median value, and/or a mode value) of the similarities of all the divided regions 110 included in the high similarity distribution region 114A1. In addition, the representative numerical value may be displayed outside the first display region 36. In this case, the representative numerical value need only be displayed in an aspect associated with the first lesion specifying image 118 or the border displayed in the second display region 38 (for example, an aspect using a word balloon attached to the first lesion specifying image 118 or the border). In addition, an identifier indicating a degree of similarity may be used instead of the numerical value indicating the representative similarity.

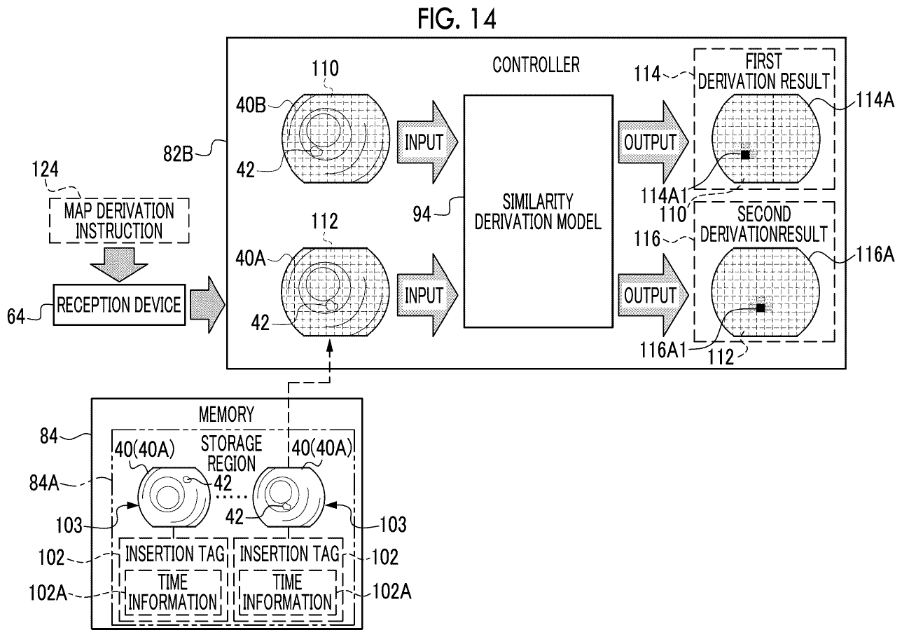

In the first embodiment, a form example is described in which the similarity is derived on a condition that the removal tag-assigned frame 106 is generated, but the present disclosure is not limited to this. For example, as shown in FIG. 14, the map 114A and/or the map 116A may be derived in response to a map derivation instruction 124 given from the outside (for example, the doctor 12). The map derivation instruction 124 is an instruction for the controller 82B to derive the map 114A and/or 116A. In the example shown in FIG. 14, the controller 82B derives the maps 114A and 116A by using the similarity derivation model 94 on a condition that the map derivation instruction 124 is received by the reception device 64. The map derivation instruction 124 is an example of a “first instruction” according to the present disclosure.

In this way, the map 114A and/or the map 116A are derived in response to the map derivation instruction 124 given from the outside, so that the doctor 12 can cause the controller 82B to derive the map 114A and/or the map 116A at a timing intended by the doctor 12.

Although the map derivation instruction 124 is illustrated here, a similarity derivation instruction may be used instead of the map derivation instruction 124. The similarity derivation instruction is an instruction for the controller 82B to derive the similarity. For example, in this case, the controller 82B derives the similarity (for example, the first derivation result 114 and/or the second derivation result 116) by using the similarity derivation model 94 on a condition that the similarity derivation instruction is received by the reception device 64.

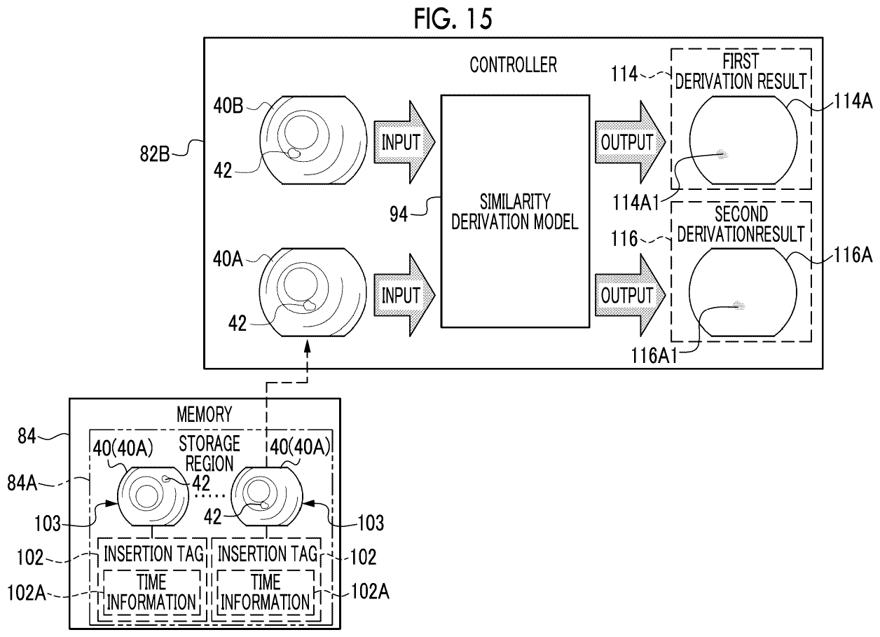

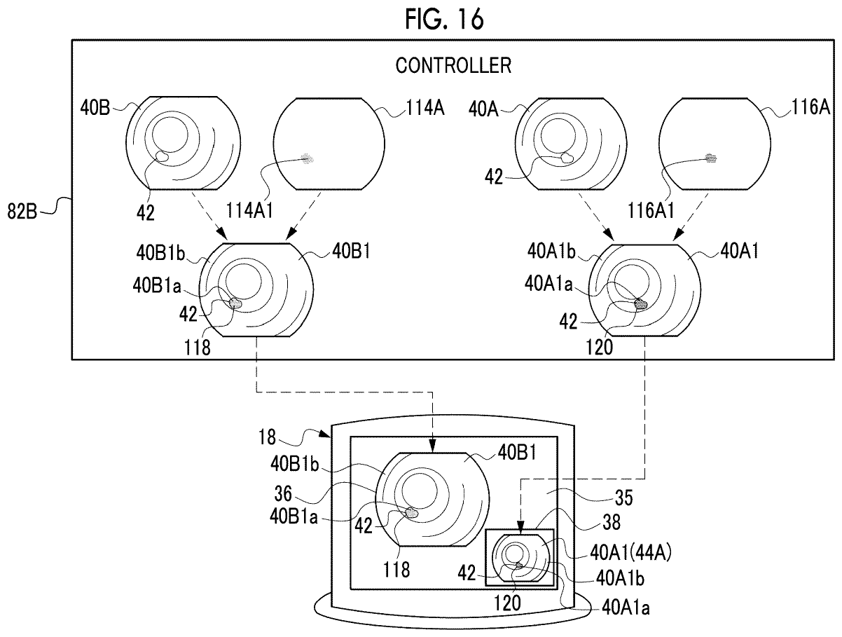

In the first embodiment, the high similarity distribution region 114A1 is defined in units of the divided region 110, and the high similarity distribution region 116A1 is defined in units of the divided region 112, but this is merely an example. For example, as shown in FIGS. 15 and 16, the high similarity distribution regions 114A1 and 116A1 may be defined in units of pixels (here, as an example, in units of one pixel). In this case, the pixel is an example of a “first divided region” and a “second divided region” according to the present disclosure. As described above, the first lesion specifying image 118 and the second lesion specifying image 120 are also defined in units of pixels by defining the high similarity distribution regions 114A1 and 116A1 in units of pixels (see FIG. 16). As a result, a shape and a size of the lesion 42 and a shape and a size of each of the first lesion specifying image 118 and the second lesion specifying image 120 can be aligned.

The high similarity distribution region 114A1 or 116A1 may be defined in units of pixels (here, as an example, in units of one pixel). By defining the high similarity distribution region 114A1 or 116A1 in units of pixels, the first lesion specifying image 118 or the second lesion specifying image 120 is also defined in units of pixels (here, as an example, in units of one pixel).

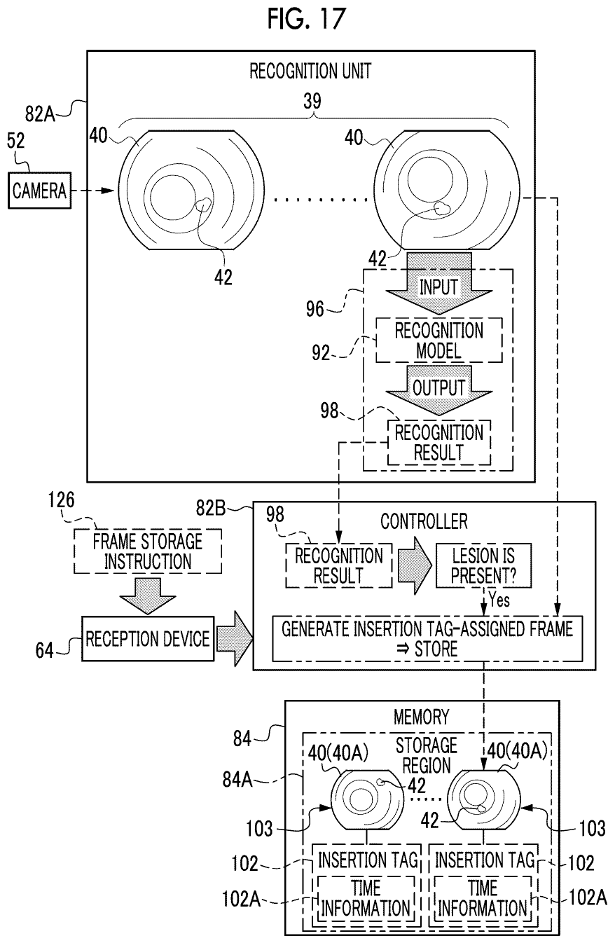

In the first embodiment, the insertion step frame 40A is stored in the storage region 84A on a condition that the lesion 42 is recognized by the recognition unit 82A, but this is merely an example. For example, as shown in FIG. 17, the insertion step frame 40A (in the example shown in FIG. 17, the insertion tag-assigned frame 103) may be stored in the storage region 84A in response to a frame storage instruction 126 given from the outside (for example, the doctor 12). The frame storage instruction 126 is an example of a “third instruction” according to the present disclosure.

The frame storage instruction 126 is an instruction to store the insertion step frame 40A in the storage region 84A. In the example shown in FIG. 17, the controller 82B stores the insertion step frame 40A (in the example shown in FIG. 17, the insertion tag-assigned frame 103) in the storage region 84A on a condition that the frame storage instruction 126 is received by the reception device 64.

In this way, the insertion step frame 40A is stored in the storage region 84A in response to the frame storage instruction 126 given from the outside, so that the doctor 12 can store the insertion step frame 40A in the storage region 84A at a timing intended by the doctor 12. In addition, the number of frames of the insertion step frames 40A stored in the storage region 84A can be reduced as compared to a case where the insertion step frame 40A is stored in the storage region 84A each time the lesion 42 is recognized. As a result, since the number of frames of the insertion step frames 40A to be input to the similarity derivation model 94 in pairs with the removal step frames 40B can be reduced, an overall load required for the derivation of the similarity can be reduced. Further, since the number of frames of the insertion step frames 40A stored in the storage region 84A can be reduced, the number of the frame sets 122 can also be reduced. As a result, it is possible to reduce an overall load required for the generation of the frame sets 122 and to suppress shortage of a capacity of the storage 86 due to the frame sets 122 being stored in the storage 86.

In addition, in the example shown in FIG. 17, the insertion tag-assigned frame 103 is stored in the storage region 84A, but the insertion tag-assigned frame 103 does not need to be stored in the storage region 84A forever. For example, in a case where the insertion tag-assigned frame 103 is used for deriving a high similarity using the similarity derivation model 94 by the controller 82B (for example, in a case where the insertion step frame 40A included in the insertion tag-assigned frame 103 is input to the similarity derivation model 94), the insertion tag-assigned frame 103 including the insertion step frame 40A used for deriving the high similarity may be erased from the storage region 84A by the controller 82B. The high similarity refers to a similarity exceeding a threshold value. In this case, for example, the insertion tag-assigned frame 103 including the insertion step frame 40A used for generating the map 116A having the high similarity distribution region 116A1 (that is, the insertion tag-assigned frame 103 including the insertion step frame 40A in which the same lesion 42 as the lesion 42 shown in the removal step frame 40B is shown) may be erased from the storage region 84A by the controller 82B. In addition, for example, the insertion tag-assigned frame 103 used for generating the frame set 122 may be erased from the storage region 84A by the controller 82B on a condition that the frame set 122 is generated by the controller 82B.

In this way, the controller 82B can specify whether or not the same lesion 42 as the lesion 42 found in the insertion step is not visually recognized by the doctor 12 through the removal step combined frame 40B1 displayed on the screen 35 in the removal step by determining whether or not the insertion tag-assigned frame 103 remains in the storage region 84A. For example, in a case where the insertion tag-assigned frame 103 remains in the storage region 84A, the controller 82B determines that the same lesion 42 as the lesion 42 found in the insertion step is not visually recognized by the doctor 12 through the removal step combined frame 40B1 displayed on the screen 35 in the removal step. In addition, for example, in a case where all the insertion tag-assigned frames 103 are erased from the storage region 84A so that the insertion tag-assigned frame 103 does not remain in the storage region 84A, the controller 82B determines that the same lesion 42 as the lesion 42 found in the insertion step is visually recognized by the doctor 12 through the removal step combined frame 40B1 displayed on the screen 35 in the removal step. These determination results may be displayed on the screen 35, whereby the doctor 12 can easily determine whether or not the lesion 42, which is seen in the insertion step, is also seen in the removal step.

In the first embodiment, a form example is described in which the insertion tag-assigned frame 103 is stored in the storage region 84A, but this is merely an example. The insertion step frame 40A may be stored in the storage region 84A.

In the first embodiment, a form example is described in which the removal tag-assigned frame 106 is generated by the controller 82B, but this is merely an example. For example, the controller 82B may derive the similarity from the similarity derivation model 94 by using the removal step frame 40B in which the lesion 42 is shown without generating the removal tag-assigned frame 106.

In the first embodiment, a form example is described in which the frame set 122 is generated by associating the insertion tag-assigned frame 103 and the removal tag-assigned frame 106 with each other, but this is merely an example. For example, the insertion step frame 40A and the removal step frame 40B may be associated with each other. The frame set obtained by associating the insertion step frame 40A and the removal step frame 40B with each other is output to a predetermined output destination (for example, the storage 86 and/or the server) by the controller 82B.

In the first embodiment, a form example is described in which the removal step combined frame 40B1 is displayed in the first display region 36 and the insertion step combined frame 40A1 is displayed in the second display region 38. However, the removal step combined frame 40B1 and the insertion step combined frame 40A1 may be displayed in one display region.

In the first embodiment, the display device 18 is illustrated as the output destination of the insertion step combined frame 40A1 and the removal step combined frame 40B1, but this is merely an example. The output destination of the insertion step combined frame 40A1 and/or the removal step combined frame 40B1 may be a storage region such as the storage 86, or may be a printer.

### Second Embodiment

In the first embodiment, a form example is described in which the removal step frame 40B is divided according to the removal step frame division rule, and the insertion step frame 40A is divided according to the insertion step frame division rule. However, in the second embodiment, a form example will be described in which a region showing the lesion 42 in the removal step frame 40B is divided, and a region showing the lesion 42 in the insertion step frame 40A is divided. In the second embodiment, the same constituents as those in the first embodiment are denoted by the same references, and the descriptions thereof will not be repeated. In addition, the descriptions regarding FIGS. 12 to 17 (for example, form examples shown in FIGS. 12 to 17 and modification examples from the form examples shown in FIGS. 12 to 17) are also applicable to the second embodiment.

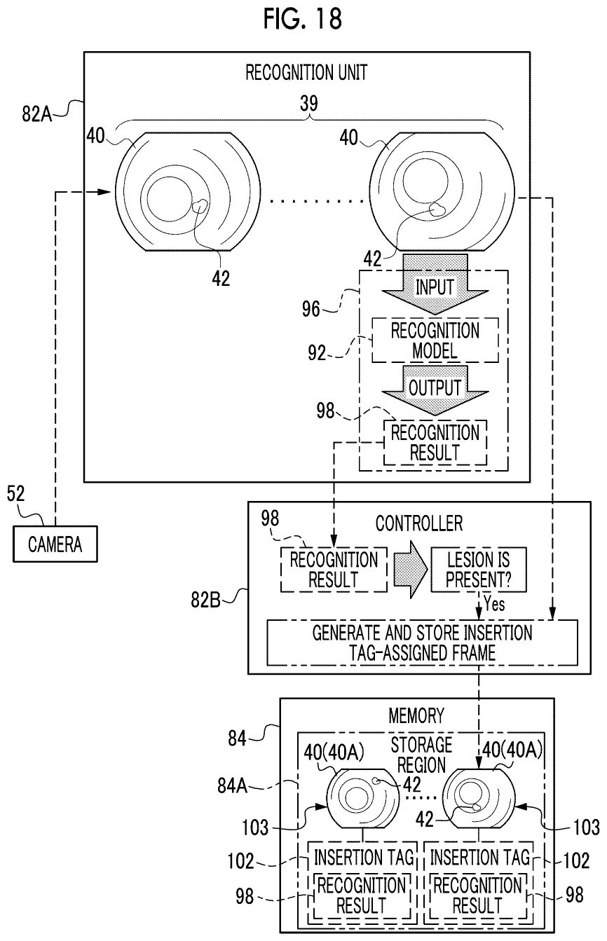

FIG. 18 is a conceptual diagram showing an example of processing contents of the recognition unit 82A and the controller 82B in the insertion step. As shown in FIG. 18, the insertion tag 102 included in the insertion tag-assigned frame 103 stored in the storage region 84A by the controller 82B includes the recognition result 98. The recognition result 98 included in the insertion tag 102 is the recognition result 98 output from the recognition model 92 by inputting the insertion step frame 40A associated with the insertion tag 102 to the recognition model 92.

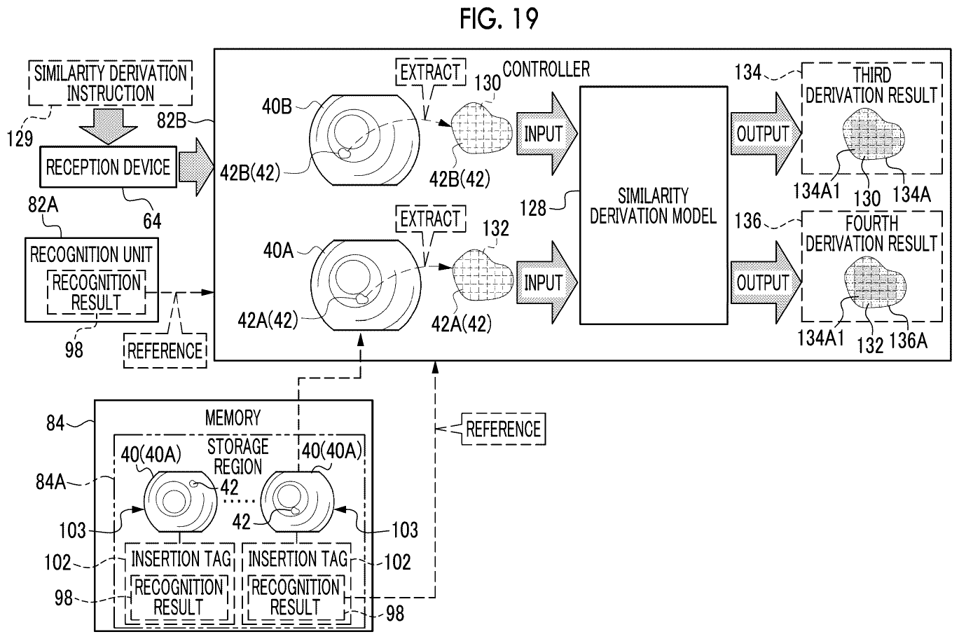

FIG. 19 is a conceptual diagram showing an example of processing contents of deriving the similarity between the lesion 42 included in the insertion step frame 40A and the lesion 42 included in the removal step frame 40B. As shown in FIG. 19, the controller 82B derives a similarity which is a degree to which the lesion 42 shown in the removal step frame 40B and the lesion 42 shown in the insertion step frame 40A are similar to each other, in response to the similarity derivation instruction 129 given from the outside (for example, the doctor 12). The similarity derivation instruction 129 is an instruction for the controller 82B to derive the similarity. In the example shown in FIG. 19, the controller 82B derives the similarity by using a similarity derivation model 128 on a condition that the similarity derivation instruction 129 is received by the reception device 64. The similarity derivation instruction 129 is an example of a “second instruction” according to the present disclosure.

The controller 82B extracts a removal step lesion region 42B, which is an image region showing the lesion 42 shown in the removal step frame 40B, from the removal step frame 40B with reference to the recognition result 98 corresponding to the removal step frame 40B (that is, the recognition result 98 obtained by performing the recognition process 96 on the removal step frame 40B) on the premise that the similarity is derived by using the similarity derivation model 128. The removal step lesion region 42B is a feature region recognized by performing the recognition process 96 as a region having features determined in advance.

In addition, the controller 82B extracts an insertion step lesion region 42A, which is an image region showing the lesion 42 shown in the unprocessed insertion step frame 40A, from the insertion step frame 40A with reference to the recognition result 98 corresponding to the unprocessed insertion step frame 40A (that is, the recognition result 98 in the insertion tag 102 associated with the insertion step frame 40A included in the insertion tag-assigned frame 103 stored in the storage region 84A) on the premise that the similarity is derived by using the similarity derivation model 128. The insertion step lesion region 42A is a feature region recognized by performing the recognition process 96 as a region having features determined in advance.

The removal step lesion region 42B has a plurality of divided regions 130. The plurality of divided regions 130 are obtained by dividing the removal step frame 40B according to a removal step lesion division rule. Here, the removal step lesion division rule refers to a rule for dividing the removal step lesion region 42B. Examples of the rule for dividing the removal step lesion region 42B include a rule in which a mesh formed in units of blocks (for example, vertical×horizontal=several pixels×several pixels) is applied to the removal step lesion region 42B to divide the removal step lesion region 42B into a mesh shape. In the second embodiment, the removal step lesion region 42B is an example of a “second region”, a “feature region”, and a “region in which a lesion is shown” according to the present disclosure. In addition, the removal step lesion division rule is an example of a “second rule” according to the present disclosure. In addition, the divided region 130 is an example of a “fourth divided region” according to the present disclosure.

The insertion step lesion region 42A has a plurality of divided regions 132. The plurality of divided regions 132 are obtained by dividing the insertion step lesion region 42A according to an insertion step lesion division rule. Here, the insertion step lesion division rule refers to a rule for dividing the insertion step lesion region 42A. Examples of the rule for dividing the insertion step lesion region 42A include a rule in which a mesh formed in units of blocks (for example, vertical×horizontal=several pixels×several pixels) is applied to the insertion step lesion region 42A to divide the insertion step lesion region 42A into a mesh shape. In the second embodiment, the insertion step lesion region 42A is an example of a “first region”, a “feature region”, and a “region in which a lesion is shown” according to the present disclosure. In addition, the insertion step lesion division rule is an example of a “second rule” according to the present disclosure. In addition, the divided region 132 is an example of a “third divided region” according to the present disclosure.

In the second embodiment, the removal step lesion division rule and the insertion step lesion division rule are the same rule. Therefore, shapes, sizes, and the number of the plurality of divided regions 130 and shapes, sizes, and the number of the plurality of divided regions 132 match.

In the second embodiment, the similarity derivation model 128 is used by the controller 82B. The similarity derivation model 128 is an example of “AI” according to the present disclosure. The similarity derivation model 128 is optimized by performing machine learning on the neural network using third training data. The third training data is a data set including a plurality of data (that is, a plurality of frames of data) in which third example data and third correct answer data are associated with each other.

The third example data is an image set of an image assuming the insertion step lesion region 42A (hereinafter, referred to as an “insertion step lesion example image”) and an image assuming the removal step lesion region 42B (hereinafter, referred to as a “removal step lesion example image”). First examples of the insertion step lesion example image include an image in which a lesion shown in an image obtained by actually imaging the inside of the large intestine with the camera is divided according to the insertion step lesion division rule. Second examples of the insertion step lesion example image include an image that is virtually created and is divided according to the insertion step lesion division rule. First examples of the removal step lesion example image include an image in which an image obtained by actually imaging the inside of the large intestine with the camera is divided according to the removal step lesion division rule. Second examples of the removal step lesion example image include an image that is virtually created and is divided according to the removal step lesion division rule.

The third correct answer data is correct answer data (that is, an annotation) for the third example data. The third correct answer data is associated with each of a plurality of divided regions obtained by dividing the insertion step lesion example image according to the insertion step lesion division rule (hereinafter, referred to as “insertion step lesion example divided region”), and each of a plurality of divided regions obtained by dividing the removal step lesion example image according to the removal step lesion division rule (hereinafter, referred to as “removal step lesion example divided region”). Examples of the third correct answer data include data indicating whether or not a lesion shown in the insertion step lesion example divided region and a lesion shown in the removal step lesion example divided region are the same.

The controller 82B inputs the insertion step lesion region 42A and the removal step lesion region 42B to the similarity derivation model 128. As a result, the similarity derivation model 128 derives a similarity which is a degree to which the divided region 130 and the divided region 132 are similar to each other. In the second embodiment, the similarity is derived for each of the divided regions 130 for the removal step lesion region 42B and is derived for each of the divided regions 132 for the insertion step lesion region 42A. In addition, the similarity derivation model 128 outputs a third derivation result 134 corresponding to the removal step lesion region 42B and a fourth derivation result 136 corresponding to the insertion step lesion region 42A each time a pair of the removal step lesion region 42B and the insertion step lesion region 42A is input.

The third derivation result 134 includes a similarity for each of the plurality of divided regions 130 and a map 134A showing a distribution of similarities. In the second embodiment, as the map 134A, a feature amount map in which a distribution of similarities exceeding a threshold value is represented in shading according to the similarity is used. Here, the similarity is defined as 0% to 100%, and 85% is used as the threshold value in this case. In the example shown in FIG. 19, a high similarity distribution region 134A1 is shown as a region in which the similarities exceeding the threshold value are distributed in the map 134A.

The fourth derivation result 136 includes a similarity for each of the plurality of divided regions 132 and a map 136A showing a distribution of similarities. In the second embodiment, as the map 136A, a feature amount map in which a distribution of similarities exceeding a threshold value is represented in shading according to the similarity is used. Here, the similarity is defined as 0% to 100%, and 85% is used as the threshold value in this case. In the example shown in FIG. 19, a high similarity distribution region 136A1 is shown as a region in which the similarities exceeding the threshold value are distributed in the map 136A.

The controller 82B performs the process shown in FIG. 19 until the high similarity distribution region 134A1 and high similarity distribution region 136A1 are obtained, or until derivation of a similarity between one removal step frame 40B and each of all the insertion step frames 40A stored in the storage region 84A shown in FIG. 19 (that is, derivation of a similarity using the similarity derivation model 128) is completed. The process shown in FIG. 19 refers to inference using the similarity derivation model 128 for the pair of the removal step lesion region 42B and the insertion step lesion region 42A. The inference using the similarity derivation model 128 for the pair of the removal step lesion region 42B and the insertion step lesion region 42A refers to processing in which the pair of the removal step lesion region 42B and the insertion step lesion region 42A is input to the similarity derivation model 128 to derive the similarity by the similarity derivation model 128, and the third derivation result 134 and the fourth derivation result 136 are output.

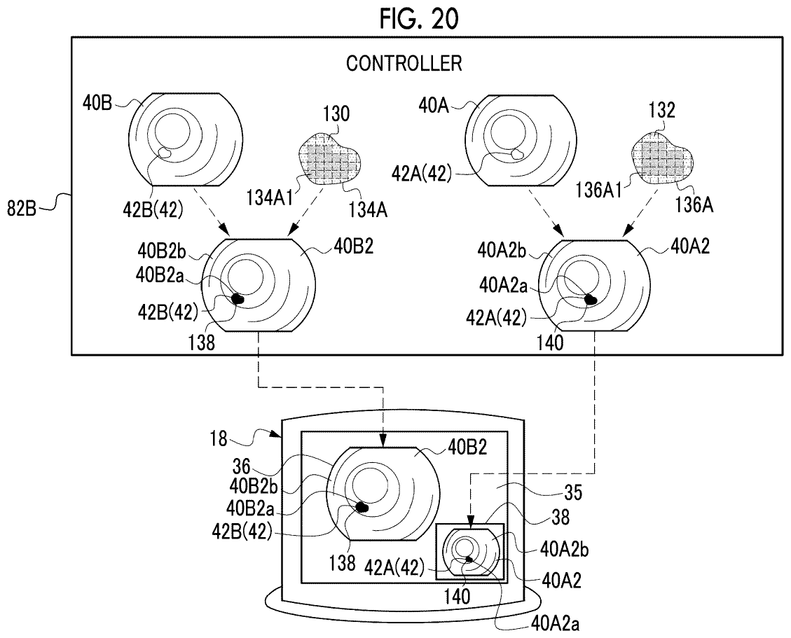

FIG. 20 is a conceptual diagram showing an example of processing contents using the removal step lesion region 42B and the map 134A by the controller 82B and processing contents using the insertion step lesion region 42A and the map 136A by the controller 82B in a case where the high similarity distribution regions 134A1 and 136A1 are obtained. As shown in FIG. 20, in a case where the map 134A including the high similarity distribution region 134A1 and the map 136A including the high similarity distribution region 136A1 are obtained from the similarity derivation model 128 (see FIG. 19), the controller 82B generates a removal step combined frame 40B2 based on the removal step frame 40B and the map 134A. Then, the controller 82B executes a removal step combined frame display process according to the second embodiment. The removal step combined frame display process according to the second embodiment is an example of a “second output process” according to the present disclosure. The removal step combined frame display process according to the second embodiment is a process of displaying the removal step combined frame 40B2 in the first display region 36. By performing the removal step combined frame display process, the removal step combined frame 40B2 is displayed in the first display region 36 in the live view method.

The removal step combined frame 40B2 is a frame in which the removal step frame 40B and a third lesion specifying image 138 corresponding to the high similarity distribution region 134A1 are combined. In the example shown in FIG. 20, a frame in which the third lesion specifying image 138 is superimposed on the removal step frame 40B is shown as the removal step combined frame 40B2.

The third lesion specifying image 138 is superimposed on a region, which is at a position corresponding to a position of the high similarity distribution region 134A1 in the map 134A, in the entire region of the removal step frame 40B. Here, as an example of the third lesion specifying image 138, the high similarity distribution region 134A1 is adopted as it is. However, this is merely an example, and an image in which the high similarity distribution region 134A1 is processed may be used. For example, an image (for example, a mark or the like) obtained by adjusting a color, a density, and/or a brightness of the high similarity distribution region 134A1 may be used. In addition, a transmittance of the third lesion specifying image 138 may be adjusted by alpha blending. Examples of the transmittance of the third lesion specifying image 138 include a transmittance to the extent that the doctor 12 can visually perceive the third lesion specifying image 138 and is not visually obstructed in observing the lesion 42 via the first display region 36 or performing a medical treatment on the lesion 42.

As described above, the removal step combined frame 40B2 obtained by superimposing the third lesion specifying image 138 on the removal step frame 40B is represented in an aspect in which a fifth image region 40B2a, which is an image region in which the lesion 42 is shown in the removal step frame 40B and a sixth image region 40B2b, which is an image region in which a portion other than the lesion 42 is shown in the removal step frame 40B, are discriminable from each other by the third lesion specifying image 138. In the example shown in FIG. 20, in the removal step combined frame 40B2, the fifth image region 40B2a is emphasized more than the sixth image region 40B2b by the third lesion specifying image 138. Here, the image region in which the lesion 42 is shown in the removal step frame 40B, that is, the fifth image region 40B2a which is a part of the removal step frame 40B is determined in units of the divided region 130. In addition, the image region in which a portion other than the lesion 42 is shown in the removal step frame 40B, that is, the sixth image region 40B2b which is a part of the removal step frame 40B is also determined in units of the divided region 130.

In the second embodiment, the fifth image region 40B2a is an example of a “second partial region” according to the present disclosure, and the sixth image region 40B2b is an example of a “second other region” according to the present disclosure.

In a case where the map 134A including the high similarity distribution region 134A1 and the map 136A including the high similarity distribution region 136A1 are obtained from the similarity derivation model 128 (see FIG. 20), the controller 82B generates the insertion step combined frame 40A2 based on the insertion step frame 40A and the map 136A. Then, the controller 82B executes an insertion step combined frame display process according to the second embodiment. The insertion step combined frame display process according to the second embodiment is an example of a “first output process” according to the present disclosure. The insertion step combined frame display process according to the second embodiment is a process of displaying the insertion step combined frame 40A2 in the second display region 38 as one of the reference images 44A.

The insertion step combined frame 40A2 is a frame in which the insertion step frame 40A and a fourth lesion specifying image 140 corresponding to the high similarity distribution region 136A1 are combined. In the example shown in FIG. 20, a frame in which the fourth lesion specifying image 140 is superimposed on the insertion step frame 40A is shown as the insertion step combined frame 40A2.

The fourth lesion specifying image 140 is superimposed on a region, which is at a position corresponding to a position of the high similarity distribution region 136A1 in the map 136A, in the entire region of the insertion step frame 40A. Here, as an example of the fourth lesion specifying image 140, the high similarity distribution region 136A1 is adopted as it is. However, this is merely an example, and an image in which the high similarity distribution region 136A1 is processed may be used. For example, an image (for example, a mark or the like) obtained by adjusting a color, a density, and/or a brightness of the high similarity distribution region 136A1 may be used. In addition, a transmittance of the fourth lesion specifying image 140 may be adjusted by alpha blending. Examples of the transmittance of the fourth lesion specifying image 140 include a transmittance to the extent that the doctor 12 can visually perceive the fourth lesion specifying image 140 and is not visually obstructed in observing the lesion 42 via the second display region 38 or performing a medical treatment on the lesion 42.

As described above, the insertion step combined frame 40A2 obtained by superimposing the fourth lesion specifying image 140 on the insertion step frame 40A is represented in an aspect in which a seventh image region 40A2a, which is an image region in which the lesion 42 is shown in the insertion step frame 40A and an eighth image region 40A2b, which is an image region in which a portion other than the lesion 42 is shown in the insertion step frame 40A, are discriminable from each other by the fourth lesion specifying image 140. In the example shown in FIG. 20, in the insertion step combined frame 40A1, the seventh image region 40A2a is emphasized more than the eighth image region 40A2b by the fourth lesion specifying image 140. Here, the image region in which the lesion 42 is shown in the insertion step frame 40A, that is, the seventh image region 40A2a which is a part of the insertion step frame 40A is determined in units of the divided region 132. In addition, the image region in which a portion other than the lesion 42 is shown in the insertion step frame 40A, that is, the eighth image region 40A2b which is a part of the insertion step frame 40A is also determined in units of the divided region 132.

In the second embodiment, the seventh image region 40A2a is an example of a “first partial region” according to the present disclosure, and the eighth image region 40A2b is an example of a “first other region” according to the present disclosure.

As described above, in the endoscope apparatus 10 according to the second embodiment, the fifth image region 40B2a on which the third lesion specifying image 138 is superimposed is defined in units of the divided region 130, and the seventh image region 40A2a on which the fourth lesion specifying image 140 is superimposed is defined in units of the divided region 132. Therefore, the fifth image region 40B2a and the seventh image region 40A2a in which the similarity is equal to or higher than the threshold value can be quickly specified as compared to a case where the entire removal step frame 40B and the entire insertion step frame 40A are divided.

In addition, in the endoscope apparatus 10 according to the second embodiment, the high similarity distribution region 134A1 in which the similarity exceeds the threshold value is extracted from the plurality of divided regions 130 according to the map 134A, and the high similarity distribution region 136A1 in which the similarity exceeds the threshold value is extracted from the plurality of divided regions 132 according to the map 136A. As a result, one or more divided regions 130 in which the similarity exceeds the threshold value can be accurately extracted as the high similarity distribution region 134A1, and one or more divided regions 132 in which the similarity exceeds the threshold value can be accurately extracted as the high similarity distribution region 136A1. In the second embodiment, the similarity is derived by an AI method by using the similarity derivation model 128. Therefore, the similarity can be accurately derived as compared to a case where the similarity is derived only based on intuition and experience of the doctor 12.

In addition, in the endoscope apparatus 10 according to the second embodiment, the similarity is derived in response to the similarity derivation instruction 129 given from the outside. As a result, the doctor 12 can cause the controller 82B to derive the similarity at a timing intended by the doctor 12.

In addition, in the endoscope apparatus 10 according to the second embodiment, the insertion step lesion region 42A is extracted from the insertion step frame 40A, and the removal step lesion region 42B is extracted from the removal step frame 40B. Both the insertion step lesion region 42A and the removal step lesion region 42B are feature regions recognized by performing the recognition process 96 as regions having features determined in advance. The feature region is a region in which the doctor 12 is highly likely to be interested. Therefore, it is possible to contribute to allowing the doctor 12 to thoroughly observe again in the removal step, via the endoscope 16, the region observed in the large intestine 28 via the endoscope 16 by the doctor 12 in the insertion step, for a region (here, an image region in which the lesion 42 is shown) for which the doctor 12 is highly likely to be interested, while the endoscope 16 is inserted into the large intestine 28.

In the second embodiment, the insertion step lesion region 42A and the removal step lesion region 42B are illustrated as the feature regions, but the present disclosure is not limited to this. For example, the feature region may be a marked region, a region in which an organ (for example, a duodenal papilla or the like) is shown, and/or a region in which the treatment tool 58 is shown.

In addition, in the endoscope apparatus 10 according to the second embodiment, a process using the recognition model 92 is performed as the recognition process 96, whereby the insertion step lesion region 42A and the removal step lesion region 42B are recognized. Therefore, the insertion step lesion region 42A and the removal step lesion region 42B can be accurately specified as compared to a case where the insertion step lesion region 42A and the removal step lesion region 42B are specified only based on intuition and experience of the doctor 12.

In addition, in the endoscope apparatus 10 according to the second embodiment, the insertion step frame 40A used in the recognition process 96 is stored in the storage region 84A on a condition that the insertion step lesion region 42A is recognized by performing the recognition process 96 in the insertion step. As a result, the insertion step frame 40A used together with the removal step frame 40B for deriving the similarity can be easily obtained.

### Other Embodiments

In each of the above-described embodiments, a form example is described in which the similarity is derived by the controller 82B, but the present disclosure is not limited to this. For example, the controller 82B may derive a difference. Here, the difference refers to a degree to which the lesion 42 shown in the insertion step frame 40A and the lesion 42 shown in the removal step frame 40B are different from each other. Also in a case where the difference is derived by the controller 82B in this way, the difference need only be derived by the AI method, and a map showing a distribution of differences need only be derived in the same manner as in each of the above-described embodiments. In each of the above-described embodiments, a form example is described in which various maps (for example, the maps 114A, 116A, 134A, and 136A) showing a distribution of similarities exceeding the threshold value are derived. However, in a case where the difference is derived, for example, various maps showing a distribution of differences of less than a threshold value (for example, 15%) different from the threshold value described in each of the above-described embodiments need only be derived instead of the various maps described in each of the above-described embodiments. As described above, even in a case where the difference is derived instead of the similarity, the same effects as those of each of the above-described embodiments can be obtained.

In each of the above-described embodiments, a form example is described in which one lesion 42 is shown in the frame 40 in order to facilitate understanding of the present disclosure, but this is merely an example. A case where a plurality of the lesions 42 are shown in the frame 40 is also considered.

In a case where such a consideration is applied to the first embodiment, since a plurality of the first image regions 40B1a are included in the removal step frame 40B, and a plurality of the third image regions 40A1a are included in the insertion step frame 40A. Therefore, the first lesion specifying image 118 is superimposed on each of the plurality of first image regions 40B1a, and the second lesion specifying image 120 is superimposed on each of the plurality of third image regions 40A1a. Here, the first lesion specifying image 118 and the second lesion specifying image 120 are displayed in a display aspect in which it is possible to visually specify whether the lesion 42 shown in which of the first image regions 40B1a included in the removal step frame 40B and the lesion 42 shown in which of the third image regions 40A1a included in the insertion step frame 40A are the same.

For example, the first lesion specifying image 118 and the second lesion specifying image 120 applied to the first image region 40B1a and the third image region 40A1a in which the same lesion 42 is shown are represented by the same color. Even in a case where the border 121 is used as shown in FIG. 13, in the same manner, the border 121 need only be displayed in a display aspect in which it is possible to visually specify whether the lesion 42 shown in which of the first image regions 40B1a included in the removal step frame 40B and the lesion 42 shown in which of the third image regions 40A1a included in the insertion step frame 40A are the same.

In addition, the same identifier (for example, an ID) may be assigned to the first image region 40B1a and the third image region 40A1a in which the same lesion 42 is shown, and the identifier assigned to the first image region 40B1a and the third image region 40A1a may be displayed in a word balloon format.

Here, a case where a plurality of the lesions 42 are shown in the frame 40 has been described with reference to the first embodiment. However, even in a case where a plurality of the lesions 42 are shown in the frame 40 in the second embodiment, in the same manner, the display aspect may be changed for each lesion 42 or the identifier determined for each lesion 42 may be displayed.

In each of the above-described embodiments, a form example is described in which the similarity between the lesion 42 shown in the insertion step frame 40A obtained in the insertion step and the lesion 42 shown in the removal step frame 40B obtained in the removal step is derived, but the present disclosure is not limited to this. For example, the similarity between the lesions 42 shown in the temporally adjacent frames 40 obtained in the insertion step or the removal step may be derived. A process using the similarity obtained as described above may be performed in the same manner as in each of the above-described embodiments. As a result, for example, in a case where the doctor 12 finds the lesion 42 after losing sight of the lesion 42 being observed due to misregistration caused by a shake of the endoscope 16 and/or body movement (that is, after the lesion 42 being observed deviates from the first display region 36), that is, in a case where the lesion 42 is found again in the first display region 36, the doctor 12 can visually specify whether or not the found lesion 42 is the same as the lesion 42 being observed before losing sight of the lesion 42.

In each of the above-described embodiments, the recognition process 96 using AI in the bounding box method has been illustrated, but the present disclosure is not limited to this. For example, a recognition process (for example, semantic segmentation, instance segmentation, and/or panoptic segmentation) using AI in a segmentation method may be performed.

In this case, the recognition model 92 is a trained model for object recognition in the segmentation method using AI. Examples of the trained model for object recognition in the segmentation method using AI include a model for semantic segmentation. Examples of the model for semantic segmentation include an encoder-decoder architecture model. Examples of the encoder-decoder architecture model include a U-Net or an HRNet.

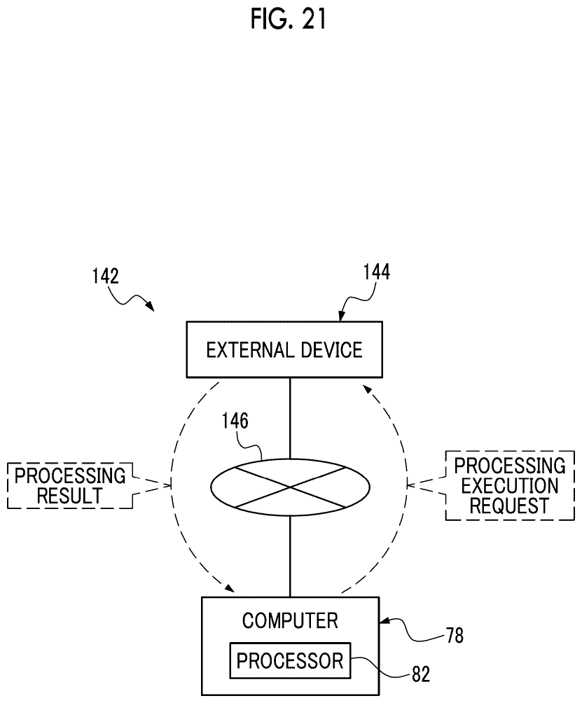

In each of the above-described embodiments, a form example is described in which the medical support process is performed by the computer 78, but the present disclosure is not limited to this. At least some of processing included in the medical support process may be performed by a device provided outside the computer 78. Hereinafter, an example of this case will be described with reference to FIG. 21.

FIG. 21 is a conceptual diagram showing an example of a configuration of an endoscope apparatus 142. The endoscope apparatus 142 is an example of an “endoscope apparatus” according to the technology of the present disclosure. The endoscope apparatus 142 is different from the endoscope apparatus 10 described in each of the above-described embodiments in that the endoscope apparatus 142 includes an external device 144.

The external device 144 is communicably connected to the computer 78 via a network 146 (for example, a WAN and/or a LAN).

Examples of the external device 144 include at least one server that directly or indirectly performs transmission and reception of data with the computer 78 via the network 146. The external device 144 receives a processing execution instruction given from the processor 82 of the computer 78 via the network 146. Then, the external device 144 executes processing according to the received processing execution instruction and transmits a processing result to the computer 78 via the network 146. In the computer 78, the processor 82 receives the processing result transmitted from the external device 144 via the network 146 and executes a process using the received processing result.

Examples of the processing execution instruction include an instruction for the external device 144 to execute at least a part of the medical support process. First examples of at least a part (that is, processing executed by the external device 144) of the medical support process include the recognition process 96. In this case, the external device 144 executes the recognition process 96 in response to the processing execution instruction given from the processor 82 via the network 146 and transmits the recognition result 98 to the computer 78 via the network 146. In the computer 78, the processor 82 receives the recognition result 98 and executes the same processing as in each of the above-described embodiments by using the received recognition result 98.

Second examples of at least a part of the medical support process (that is, processing executed by the external device 144) include processing by the controller 82B. In this case, the external device 144 executes the processing by the controller 82B in response to the processing execution instruction given from the processor 82 via the network 146, and transmits a processing result (for example, the insertion step combined frame 40A1, the insertion step combined frame 40A2, the removal step combined frame 40B1, and/or the removal step combined frame 40B2, and the like) to the computer 78 via the network 146. In the computer 78, the processor 82 receives the processing result (for example, the insertion step combined frame 40A1, the insertion step combined frame 40A2, the removal step combined frame 40B1, and/or the removal step combined frame 40B2, and the like), and executes the same processing as in each of the above-described embodiments by using the received processing result.

For example, the external device 144 is realized by cloud computing. It should be noted that the cloud computing is merely an example, and the external device 144 may be realized by network computing such as fog computing, edge computing, or grid computing. Instead of the server, at least one personal computer or the like may be used as the external device 144. In addition, a computing device having a communication function equipped with a plurality of types of AI functions may be used.

In each of the above-described embodiments, a form example is described in which the medical support program 90 is stored in the storage 86, but the present disclosure is not limited to this. For example, the medical support program 90 may be stored in a portable computer-readable non-transitory storage medium, such as an SSD or a USB memory. The medical support program 90 stored in the non-transitory storage medium is installed in the computer 78 of the endoscope apparatus 10. The processor 82 executes the medical support process according to the medical support program 90.

In addition, the medical support program 90 may be stored in a storage device of another computer, server, or the like connected to the endoscope apparatus 10 via a network, and the medical support program 90 may be downloaded and installed in the computer 78 in response to a request from the endoscope apparatus 10.

It is not necessary to store all the medical support programs 90 in a storage device of another computer, server device, or the like connected to the endoscope apparatus 10 or to store all the medical support programs 90 in the storage 86, and a part of the medical support programs 90 may be stored.

The following various processors can be used as hardware resources for executing the medical support process. Examples of the processor include a CPU which is a general-purpose processor that executes software, that is, a program, to function as the hardware resource executing the medical support process. In addition, examples of the processor include a dedicated electric circuit which is a processor having a circuit configuration specially designed for executing specific processing, such as an FPGA, a PLD, or an ASIC. A memory is incorporated in or connected to any processor, and any processor executes the medical support process using the memory.

The hardware resource for executing the medical support process may be configured by one of the various processors or by a combination of two or more processors of the same type or different types (for example, a combination of a plurality of FPGAs or a combination of a CPU and an FPGA). Further, the hardware resource for executing the medical support process may be one processor.

As an example of the configuration using one processor, first, there is a form in which one processor is configured by a combination of one or more CPUs and software, and the processor functions as the hardware resource for executing the medical support process. Second, as typified by a SoC or the like, there is a form in which a processor that realizes all functions of a system including a plurality of hardware resources executing the medical support process with one IC chip is used. As described above, the medical support process is realized using one or more of the various processors as the hardware resource.

Further, as a hardware structure of these various processors, more specifically, an electrical circuit in which circuit elements such as semiconductor elements are combined can be used. Further, the above-described medical support process is only an example. Therefore, it is needless to say that unnecessary steps may be deleted, new steps may be added, or a processing order may be changed without departing from the gist of the present disclosure.

The above-described contents and illustrated contents are detailed descriptions of parts related to the present disclosure, and are merely examples of the present disclosure. For example, the above descriptions related to configurations, functions, operations, and advantageous effects are descriptions related to examples of configurations, functions, operations, and advantageous effects of the parts related to the present disclosure. Therefore, it is needless to say that unnecessary parts may be deleted, or new elements may be added or replaced with respect to the above-described contents and illustrated contents without departing from the gist of the present disclosure. In order to avoid complications and easily understand the parts according to the present disclosure, in the above-described contents and illustrated contents, common technical knowledge and the like that do not need to be described to implement the present disclosure are not described.

All documents, patent applications, and technical standards described in the present specification are incorporated in the present specification by reference to the same extent as in a case where each document, patent application, and technical standard are specifically and individually noted to be incorporated by reference.

What is claimed is:

1. A medical support device comprising:
a processor,
wherein the processor is configured to:
acquire a first medical image obtained by imaging an inside of a luminal organ with an endoscope and a second medical image obtained temporally later than the first medical image by imaging the inside of the luminal organ with the endoscope while the endoscope is inserted into the luminal organ; and
execute a first output process of outputting the first medical image and/or a second output process of outputting the second medical image in a case where a similarity between a first partial region that is a part of the first medical image and a second partial region that is a part of the second medical image exceeds a threshold value,
the first medical image output by executing the first output process is an image represented in an aspect in which the first partial region and a first other region that is another region in the first medical image are discriminable from each other, and
the second medical image output by executing the second output process is an image represented in an aspect in which the second partial region and a second other region that is another region in the second medical image are discriminable from each other.

2. The medical support device according to claim 1,
wherein the first medical image output by executing the first output process is an image in which the first partial region is emphasized more than the first other region, and
the second medical image output by executing the second output process is an image in which the second partial region is emphasized more than the second other region.

3. The medical support device according to claim 1,
wherein the first medical image is an image obtained in an insertion step in which the endoscope is inserted into the luminal organ, and
the second medical image is an image obtained in a removal step in which the endoscope is removed from the luminal organ.

4. The medical support device according to claim 1,
wherein the first partial region is at least one of a plurality of first divided regions obtained by dividing the first medical image according to a first rule, and
the second partial region is at least one of a plurality of second divided regions obtained by dividing the second medical image according to the first rule.

5. The medical support device according to claim 4,
wherein the processor is further configured to:
derive a map showing a distribution of the similarities between the plurality of first divided regions and the plurality of second divided regions based on the first medical image and the second medical image; and
extract, from the plurality of first divided regions and the plurality of second divided regions, the first divided region and the second divided region in which the similarity exceeds the threshold value according to the derived map, as the first partial region and the second partial region in which the similarity exceeds the threshold value.

6. The medical support device according to claim 5,
wherein the processor derives the map by using AI.

7. The medical support device according to claim 5,
wherein the map is derived according to a first instruction given from an outside.

8. The medical support device according to claim 1,
wherein the first partial region is at least one of a plurality of third divided regions obtained by dividing a first region that is a part of the first medical image according to a second rule, and
the second partial region is at least one of a plurality of fourth divided regions obtained by dividing a second region that is a part of the second medical image according to the second rule.

9. The medical support device according to claim 8,
wherein the processor is further configured to:
derive the similarity between each of the plurality of third divided regions and each of the plurality of fourth divided regions based on each of the plurality of third divided regions and each of the plurality of fourth divided regions; and
extract, from the plurality of third divided regions and the plurality of fourth divided regions, the third divided region and the fourth divided region in which the similarity exceeds the threshold value as the first partial region and the second partial region in which the similarity exceeds the threshold value.

10. The medical support device according to claim 9,
wherein the processor derives the similarity by using AI.

11. The medical support device according to claim 10,
wherein the similarity is derived according to a second instruction given from an outside.

12. The medical support device according to claim 8,
wherein each of the first region and the second region is a feature region recognized by performing an object recognition process as a region having features determined in advance.

13. The medical support device according to claim 12,
wherein the feature region is a region in which a lesion is shown, a marked region, a region in which an organ is shown, and/or a region in which a treatment tool is shown.

14. The medical support device according to claim 12,
wherein object recognition AI is used in the object recognition process.

15. The medical support device according to claim 12,
wherein the first medical image is stored in a first storage region on a condition that the first region is recognized as the feature region by performing the object recognition process.

16. The medical support device according to claim 1,
wherein the first medical image is stored in a second storage region in response to a third instruction given from an outside.

17. The medical support device according to claim 1,
wherein the first medical image and the second medical image are images obtained by imaging a region irradiated with the same type of light in the luminal organ, by the endoscope.

18. The medical support device according to claim 1,
wherein the first medical image output by executing the first output process is displayed on a first screen, and
the second medical image output by executing the second output process is displayed on a second screen.

19. A medical support device comprising:
a processor,
wherein the processor is configured to:
acquire a first medical image obtained by imaging an inside of a luminal organ with an endoscope and a second medical image obtained temporally later than the first medical image by imaging the inside of the luminal organ with the endoscope while the endoscope is inserted into the luminal organ; and
execute a first output process of outputting the first medical image and/or a second output process of outputting the second medical image in a case where a difference between a first partial region that is a part of the first medical image and a second partial region that is a part of the second medical image is less than a threshold value,
the first medical image output by executing the first output process is an image represented in an aspect in which the first partial region and a first other region that is another region in the first medical image are discriminable from each other, and
the second medical image output by executing the second output process is an image represented in an aspect in which the second partial region and a second other region that is another region in the second medical image are discriminable from each other.

20. An endoscope apparatus comprising:
the medical support device according to claim 1; and
the endoscope.

21. A medical support method comprising:
acquiring a first medical image obtained by imaging an inside of a luminal organ with an endoscope and a second medical image obtained temporally later than the first medical image by imaging the inside of the luminal organ with the endoscope while the endoscope is inserted into the luminal organ; and
executing a first output process of outputting the first medical image and/or a second output process of outputting the second medical image in a case where a similarity between a first partial region that is a part of the first medical image and a second partial region that is a part of the second medical image exceeds a threshold value,
wherein the first medical image output by executing the first output process is an image represented in an aspect in which the first partial region and a first other region that is another region in the first medical image are discriminable from each other, and
the second medical image output by executing the second output process is an image represented in an aspect in which the second partial region and a second other region that is another region in the second medical image are discriminable from each other.

22. A non-transitory computer-readable storage medium storing a program executable by a computer to execute a medical support process, the medical support process comprising:
acquiring a first medical image obtained by imaging an inside of a luminal organ with an endoscope and a second medical image obtained temporally later than the first medical image by imaging the inside of the luminal organ with the endoscope while the endoscope is inserted into the luminal organ; and
executing a first output process of outputting the first medical image and/or a second output process of outputting the second medical image in a case where a similarity between a first partial region that is a part of the first medical image and a second partial region that is a part of the second medical image exceeds a threshold value,
wherein the first medical image output by executing the first output process is an image represented in an aspect in which the first partial region and a first other region that is another region in the first medical image are discriminable from each other, and
the second medical image output by executing the second output process is an image represented in an aspect in which the second partial region and a second other region that is another region in the second medical image are discriminable from each other.
